(19) United States
(12) Patent Application Publication
(10) Pub. No.: US 2018/0186815 A1
(43) Pub. Date: Jul. 5, 2018

(54) PHARMACEUTICAL COMPOSITIONS, METHODS FOR THEIR PREPARATION AND THEIR USE IN THE TREATMENT OF CANCER

(71) Applicant: Urifer Ltd., Raanana (IL)

(72) Inventors: Uri NIR, Moshav Gamzo (IL); Sally SHPUNGIN, Ganei Tikva (IL)

(21) Appl. No.: 15/857,819

(22) Filed: Dec. 29, 2017

(30) Foreign Application Priority Data
Dec. 29, 2016 (IL) 249860

(51) Int. Cl. C07D 513/04; A61P 35/00

(52) U.S. Cl. CPC C07D 513/04; A61P 35/00

(57) ABSTRACT

The present application is directed to pharmaceutical compositions comprising derivatives of 6-(4-substitutedphenyl)-2-(substituted piperidine)imidazo[2,1-b][1,3,4]thiadiazol-2-yl], to a method of treating cancer using these compositions and to processes for preparing these derivatives.

### TECHNOLOGICAL FIELD

This invention relates to pharmaceutical compositions and methods for the treatment of cancer.

### BACKGROUND ART

The following references are considered to be relevant for an understanding of the invention.

### REFERENCES

Allard P, Zoubeidi A, Nguyen L T, Tessier S, Tanguay S, Chevrette M, Aprikian A and Chevalier S. (2000). Mol. Cell. Endocrinol., 159, 63-77.
Berridge, M. V., Herst, P. M., and Tan, A. S. (2005). Tetrazolium dyes as tools in cell biology: new insights into their cellular reduction. Biotechnol. Annu. Rev. 11, 127-152]
Craig A W and Greer P A. (2002). Mol. Cell. Biol., 22, 6363-6374.
Craig A W, Zirngibl R, Williams K, Cole L A and Greer P A. (2001). Mol. Cell. Biol., 21, 603-613.
Greer P. (2002). Nat. Rev. Mol. Cell Biol., 3, 278-289.
Hao Q-L, Heisterkamp N and Groffen J. (1989). Mol. Cell. Biol., 9, 1587-1593.
Kim L and Wong T W. (1998). J. Biol. Chem., 273, 23542-23548.
Orlovsky K, Ben-Dor I, Priel-Halachmi S, Malovany H and Nir U. (2000). Biochemistry, 39, 11084-11091.
Penhallow R C, Class K, Sonoda H, Bolen J B and Rowley R B. (1995). J. Biol. Chem., 270, 23362-23365.
Pasder, O., Shpungin, S., Salem, Y., Makovsky, A., Vilchick, S., Michaeli, S., Malovani, H. and Nir, U. (2006) Oncogene, 25, 4194-4206.
Pasder, O., Salem, Y., Yaffe, E., Shpungin, S. and Nir, U. (2007) Drugs of the Future, 32, 61-70.

### BACKGROUND

Fer is an intracellular tyrosine kinase that resides in both the cytoplasm and nucleus of mammalian cells and is activated by growth factors such as EGF and PDGF in fibroblastic cells (Kim and Wong, 1998), and by occupation of the Fcγ receptor in mast cells (Penhallow et al., 1995). Although present in a wide variety of tissues and cells, the functional role of Fer has been elucidated mainly in cells which carry out innate immune responses (Craig and Greer, 2002; Greer, 2002). Mice devoid of an active Fer develop normally and the proliferation of fibroblasts derived from these mice is not impaired in vitro (Craig et al., 2001).

Fer was detected in all human malignant cell lines analyzed (Hao et al., 1989; Orlovsky et al., 2000) and its levels in malignant prostate tumors are significantly higher than those detected in benign prostate tumors (Allard et al., 2000). Furthermore, down-regulation of Fer impaired the proliferation of prostate and breast carcinoma cells (Pasder et al., 2006) and abolished the ability of prostate carcinoma PC3 cells to form colonies in soft agar (Allard et al., 2000). U.S. patent application Ser. No. 10/486,101 having Publication Number 20050063973 discloses short interfering RNA (siRNA) molecules directed to sequences of the fer gene. These siRNA molecules were found to inhibit the growth of PC3 cells and to arrest tumor growth in an animal model (Pasder et al., 2007).

### General Description

In its first aspect, the present invention provides pharmaceutical compositions. A pharmaceutical composition according to the invention comprises as an active ingredient a compound of formulae (I) or (II) or a pharmaceutically acceptable salt thereof:

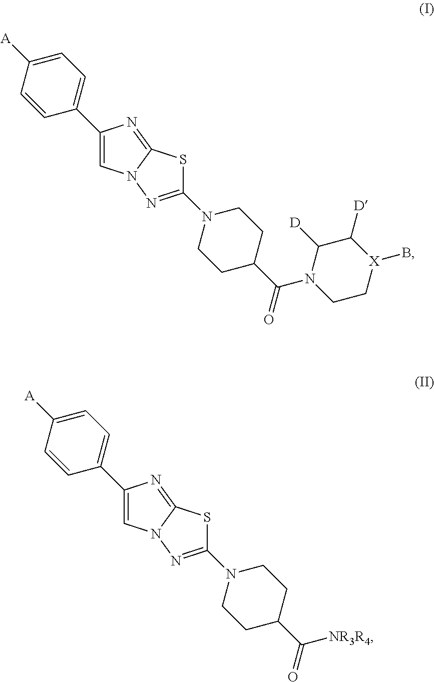

wherein:

X is N or C;

A is: H, halogen; a straight or branched C₁-C₅ alkyl, NR₁R₂, or a C₅₋₈-heterocyclyl or heteroaryl having 1, 2 or 3 heteroatoms selected from N, O, S, that may be substituted by a straight or branched C₁-C₅ alkyl;

B is H, straight or branched C₁-C₅, (CH₂)ₙ—NR₁R₂, (CH₂)ₙ—C(═O)—NR₁R₂(CH₂)ₙ—NR₁R₂; a C₅₋₇-heterocyclyl or heteroaryl having 1, 2 or 3 heteroatoms selected from N, O, S, that may be substituted by straight or branched C₁-C₅ alkyl, (CH₂)ₙ—C₅₋₇-heterocyclyl or heteroaryl, or (CH₂)ₙ—C(═O)—C₅₋₇-hetercyclyl or heteroaryl, having 1, 2 or 3 heteroatoms selected from N, O, S, that may be substituted by straight or branched C₁-C₅ alkyl;

D and D′ are independently H or (CH₂)ₙ—C₅₋₇-heterocyclyl or heteroaryl, having 1, 2 or 3 heteroatoms selected from N, O, S;

provided that, when X is C, one of D and D′ is H and the other is (CH₂)ₙ-heterocylcyl and B is H; and, when X is N, D and D′ are both H;

R₁ and R₂ are independently H, straight or branched C₁-C₅ alkyl or straight or branched C₁-C₅—OH;

R₃ is H, straight or branched C₁-C₅ alkyl, or (CH₂)ₙNR₁R₂;

R₄ is (CH₂)ₙ—C(═O)—NR₁R₂—NR₁R₂ or (CH₂)ₙ—C₅₋₇-hetercyclyl or heteroaryl having 1, 2 or 3 heteroatoms selected from N, O, S;

n is 0, 1, 2 or 3;

provided that the following compounds:

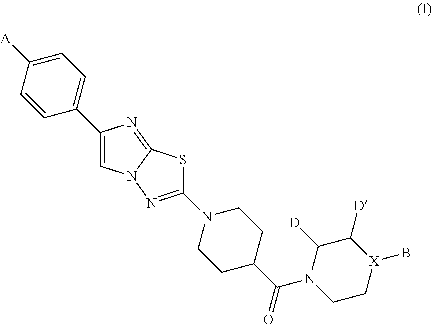

where:

(1) A is H, halogen, a straight or branched C₁-C₅ alkyl;

(2) D and D′ are H; and

(3) B is H or straight or branched C₁-C₅ are excluded.

In particular, the pharmaceutical compositions of the invention have been found to inhibit the growth of cancer cells. Without wishing to be bound by a particular theory, it is believed that the pharmaceutical compositions of the invention inhibit the expression or the activity of Fer in the treated cells.

In one preferred embodiment, the pharmaceutical composition of the invention comprises compounds, having the following formulae:

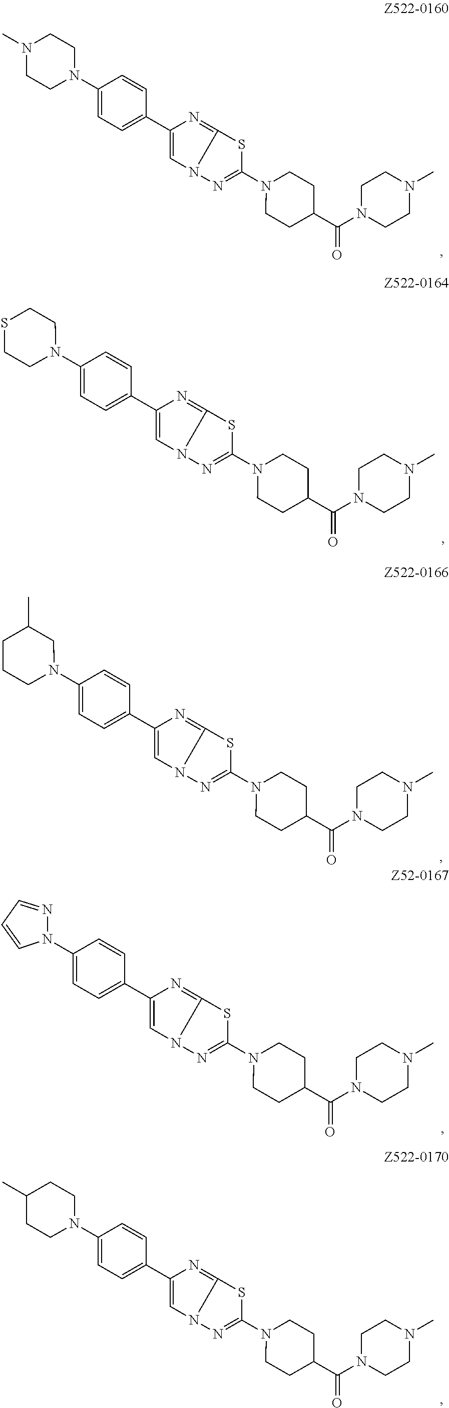

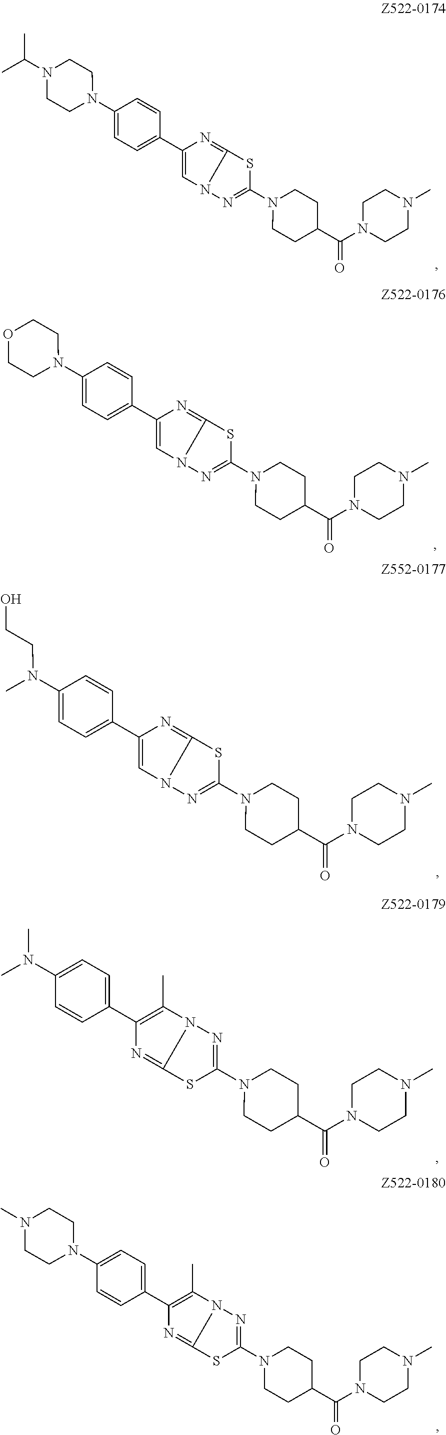

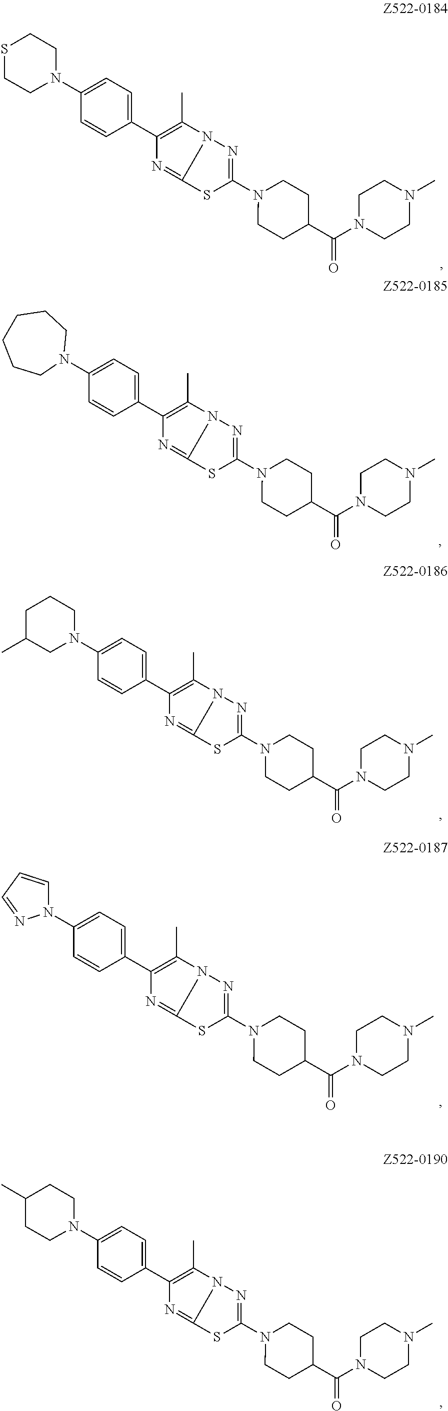

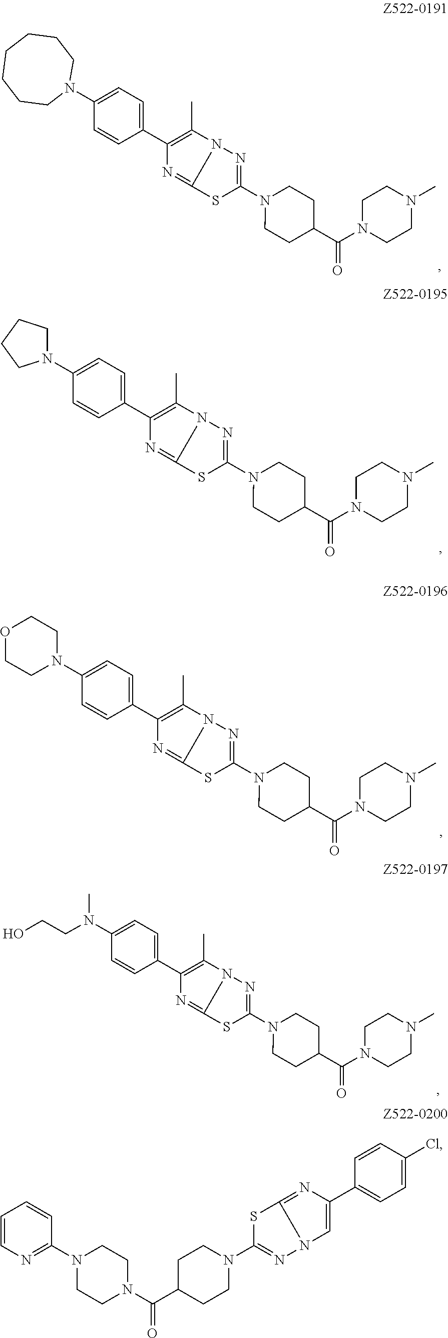 [SMILES: Cc1c(-c2ccc(N3CCCCCCC3)cc2)nc2sc(N3CCC(C(=O)N4CCN(C)CC4)CC3)nn12 ; Cc1c(-c2ccc(N3CCCC3)cc2)nc2sc(N3CCC(C(=O)N4CCN(C)CC4)CC3)nn12 ; Cc1c(-c2ccc(N3CCOCC3)cc2)nc2sc(N3CCC(C(=O)N4CCN(C)CC4)CC3)nn12 ; Cc1c(-c2ccc(N(C)CCO)cc2)nc2sc(N3CCC(C(=O)N4CCN(C)CC4)CC3)nn12 ; O=C(C1CCN(c2nn3cc(-c4ccc(Cl)cc4)nc3s2)CC1)N1CCN(c2ccccn2)CC1]

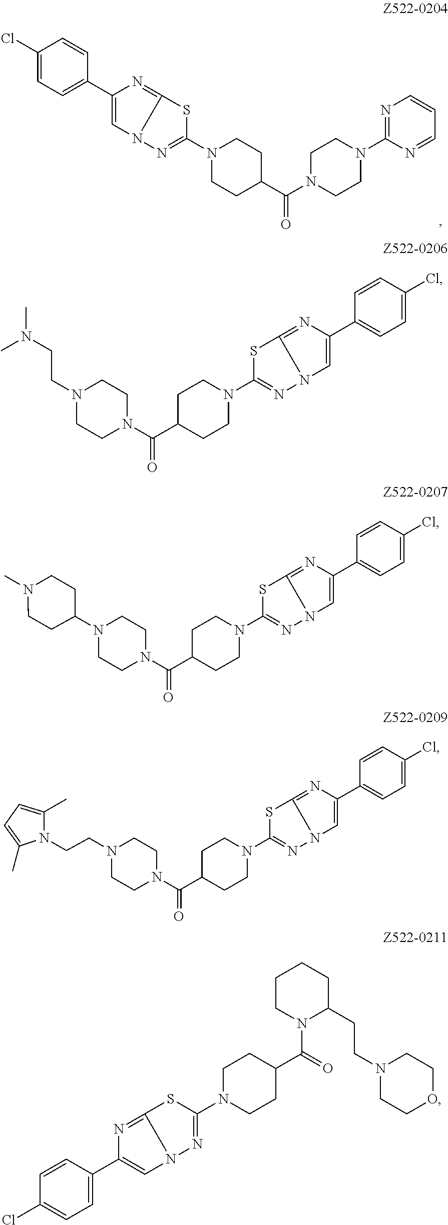 [SMILES: O=C(C1CCN(c2nn3cc(-c4ccc(Cl)cc4)nc3s2)CC1)N1CCN(c2ncccn2)CC1 ; CN(C)CCN1CCN(C(=O)C2CCN(c3nn4cc(-c5ccc(Cl)cc5)nc4s3)CC2)CC1 ; CN1CCC(N2CCN(C(=O)C3CCN(c4nn5cc(-c6ccc(Cl)cc6)nc5s4)CC3)CC2)CC1 ; Cc1ccc(C)n1CCN1CCN(C(=O)C2CCN(c3nn4cc(-c5ccc(Cl)cc5)nc4s3)CC2)CC1 ; O=C(C1CCN(c2nn3cc(-c4ccc(Cl)cc4)nc3s2)CC1)N1CCCCC1CCN1CCOCC1]

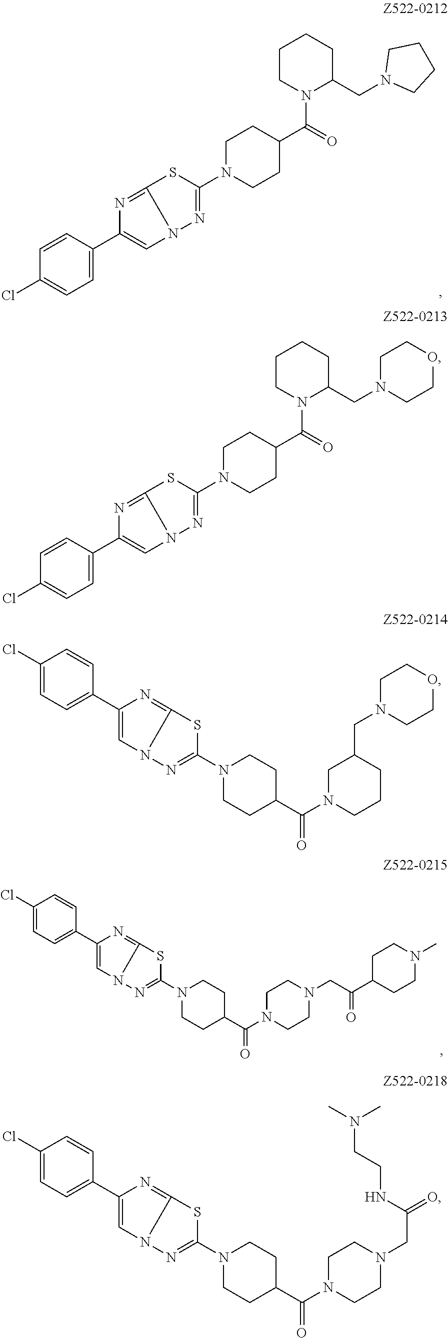 [SMILES: O=C(C1CCN(c2nn3cc(-c4ccc(Cl)cc4)nc3s2)CC1)N1CCCCC1CN1CCCC1 ; O=C(C1CCN(c2nn3cc(-c4ccc(Cl)cc4)nc3s2)CC1)N1CCCCC1CN1CCOCC1 ; O=C(C1CCN(c2nn3cc(-c4ccc(Cl)cc4)nc3s2)CC1)N1CCCC(CN2CCOCC2)C1 ; CN1CCC(C(=O)CN2CCN(C(=O)C3CCN(c4nn5cc(-c6ccc(Cl)cc6)nc5s4)CC3)CC2)CC1 ; CN(C)CCNC(=O)CN1CCN(C(=O)C2CCN(c3nn4cc(-c5ccc(Cl)cc5)nc4s3)CC2)CC1]

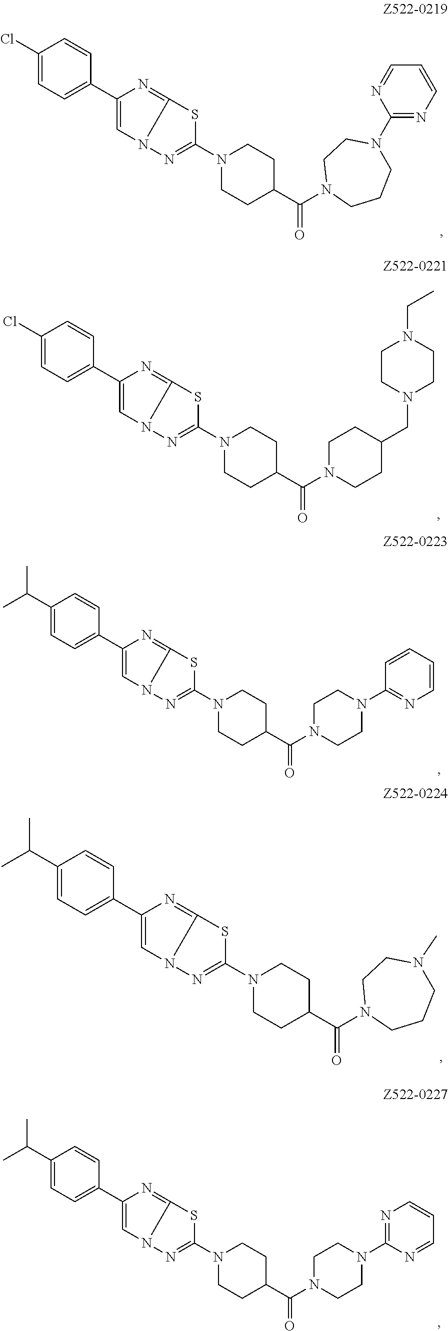

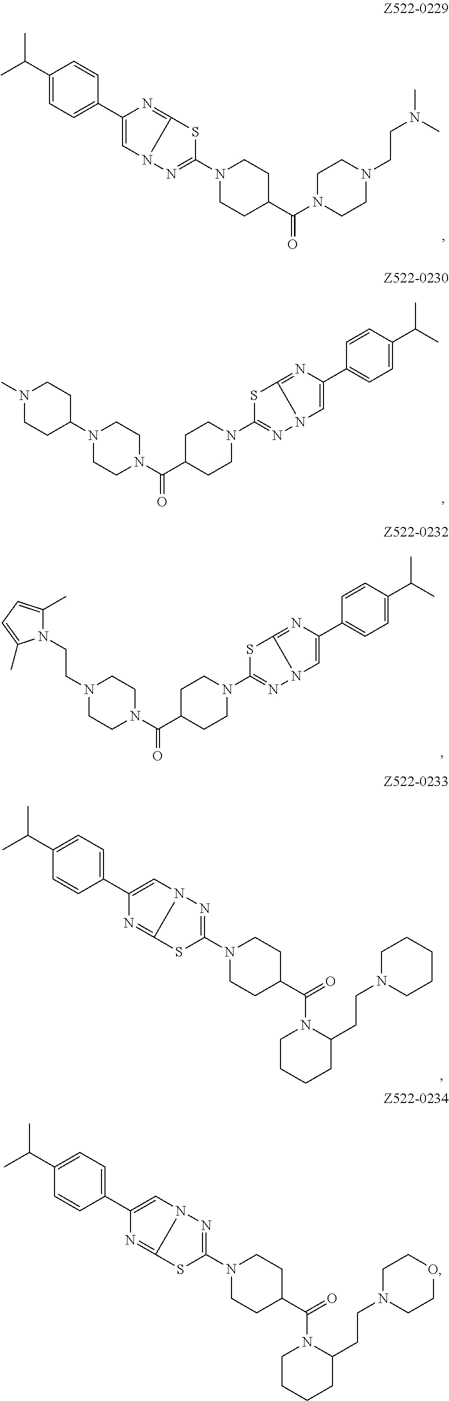

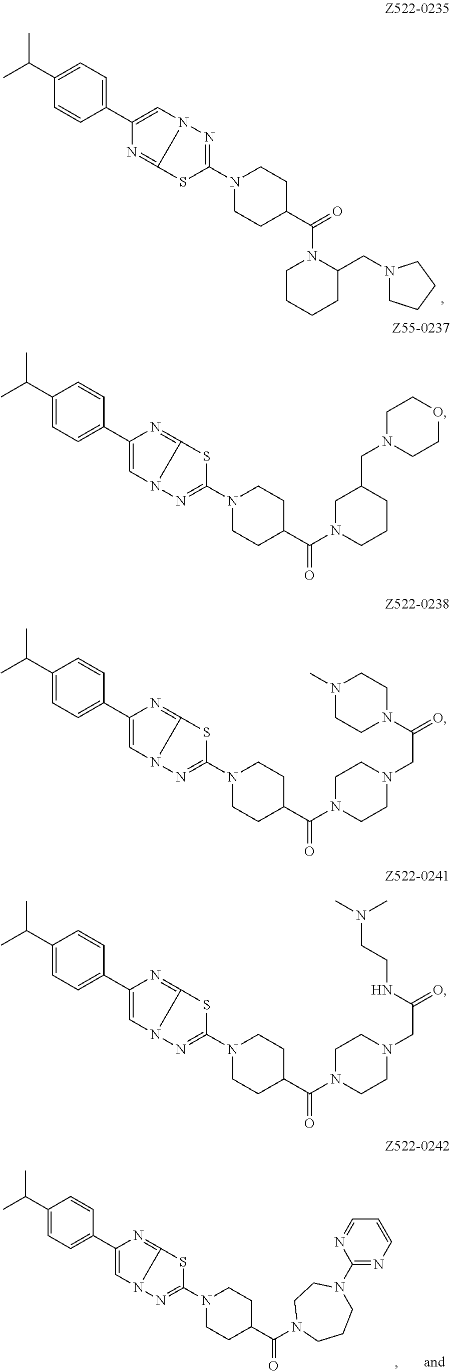 [SMILES: CC(C)c1ccc(-c2cn3nc(N4CCC(C(=O)N5CCCCC5CN5CCCC5)CC4)sc3n2)cc1 ; CC(C)c1ccc(-c2cn3nc(N4CCC(C(=O)N5CCCC(CN6CCOCC6)C5)CC4)sc3n2)cc1 ; CC(C)c1ccc(-c2cn3nc(N4CCC(C(=O)N5CCN(CC(=O)N6CCN(C)CC6)CC5)CC4)sc3n2)cc1 ; CC(C)c1ccc(-c2cn3nc(N4CCC(C(=O)N5CCN(CC(=O)NCCN(C)C)CC5)CC4)sc3n2)cc1 ; CC(C)c1ccc(-c2cn3nc(N4CCC(C(=O)N5CCCN(c6ncccn6)CC5)CC4)sc3n2)cc1]

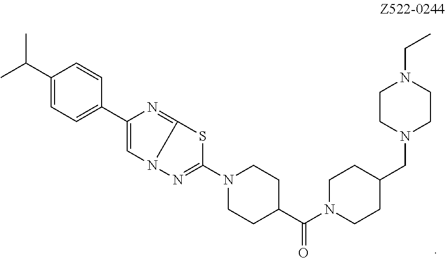 [SMILES: CCN1CCN(CC2CCN(C(=O)C3CCN(c4nn5cc(-c6ccc(C(C)C)cc6)nc5s4)CC3)CC2)CC1]

In another preferred embodiment, the pharmaceutical composition of the invention comprises compounds, having the following formulae:

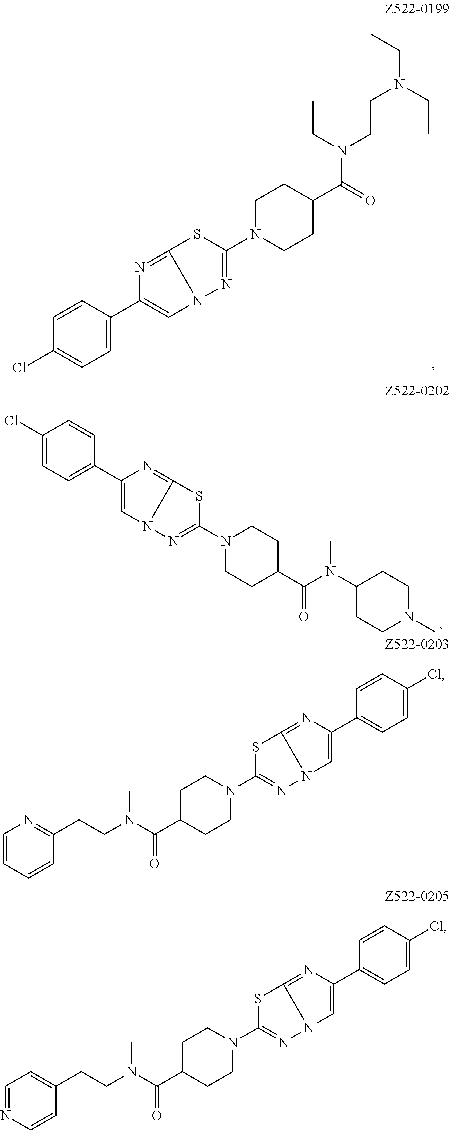

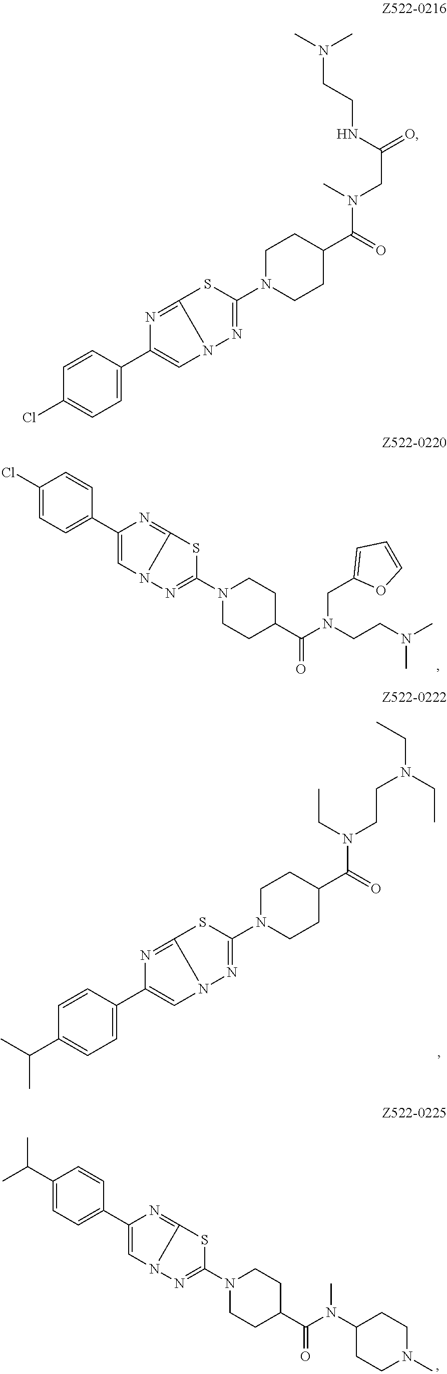

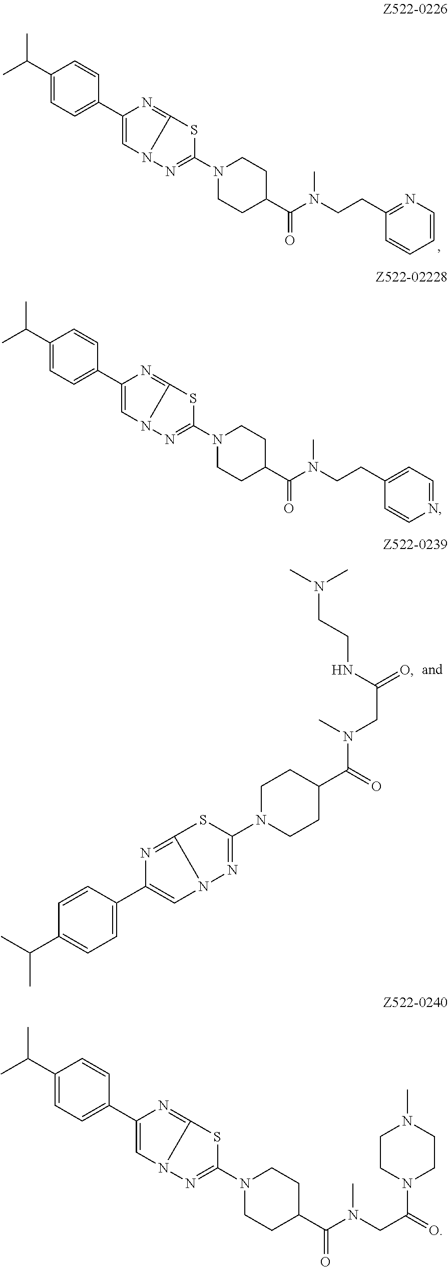

In a further embodiment, the present invention is directed the following compounds:

Z522-166, Z522-0170, Z522-186, Z522-202, Z522-0218, Z522-220, Z522-222, Z522-224, Z522-225, Z522-0234, Z522-0237, Z522-0238, Z522-0239, Z522-0241.

In a second aspect, the invention provides methods of synthesis of the compounds of the pharmaceutical composition of the invention.

In a third aspect, the invention provides a method for treating cancer. In accordance with this aspect of the invention, an individual in need of such treatment is administered an effective amount of a pharmaceutical composition of the invention. In particular the cancer that is treated is colon cancer.

### DETAILED DESCRIPTION OF EMBODIMENTS

The invention provides pharmaceutical compositions comprising as an active ingredient a compound of formulae (I) or (II) as defined.

Straight or branched C₁-C₅ alkyl means methyl, ethyl, propyl, iso-propyl, n-butyl, iso-butyl (tert-butyl), n-pentyl, iso-pentyl, neo-pentyl.

Straight or branched C₁-C₅—OH; represents a straight or branched alkyl chain having 1, 2 or 3 hydroxyl groups that may be attached to any of the carbons in the straight or branched chain.

Halogen represents chlorine, bromine or iodine.

Pharmaceutically acceptable salts of the compounds (I) and (II) are organic or inorganic pharmaceutically acceptable salts. Suitable pharmaceutically acceptable salts include basic salts of inorganic and organic acids, such as hydrochloric acid, hydrobromic acid, sulfuric acid, phosphoric acid, methanesulphonic acid, trifluoromethanesulfonic acid, benzenesulfonic acid, p-toluenesulfonic acid, 1-naphthalenesulfonic acid, 2naphthalenesulfonic acid, acetic acid, trifluoroacetic acid, malic acid, tartaric acid, citric acid, lactic acid, oxalic acid, succinic acid, fumaric acid, maleic acid, benzoic acid, salicylic acid, phenylacetic acid, and mandelic acid. In addition, pharmaceutically acceptable salts include acid salts of inorganic bases, such as salts containing alkaline cations (e.g., Li⁺Na⁺ or K⁺), alkaline earth cations (e.g., Mg⁺², Ca⁺² or Ba⁺²), the ammonium cation, as well as acid salts of organic bases, including aliphatic and aromatic substituted ammonium, and quaternary ammonium cations, such as those arising from protonation or peralkylation of triethylamine, N, N-diethylamine, N, N-dicyclohexylamine, lysine, pyridine, N, N-dimethylaminopyridine (DMAP), 1,4-diazabiclo[2.2.2] octane (DABCO), 1,5-diazabicyclo[4.3.0]non-5-ene (DBN) and 1,8-diazabicyclo[5.4.0]undec-7-ene (DBU).

The compounds of the invention were synthesized according to the following schemes:

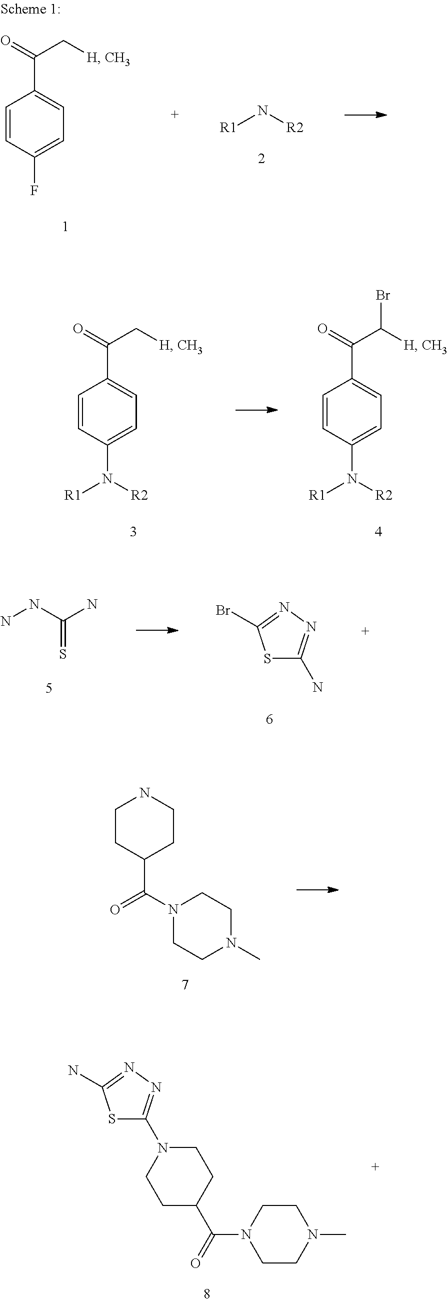

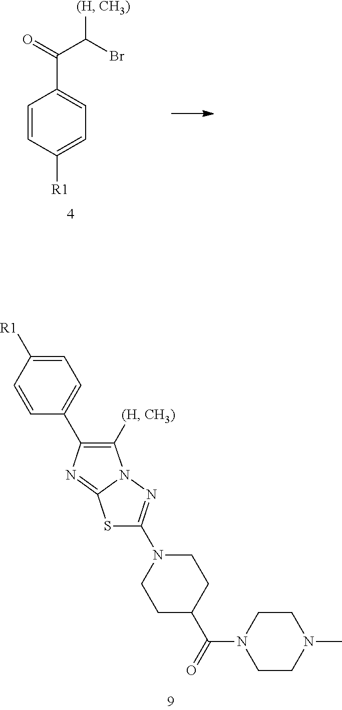

Preparation of 3:

10 mmol of 1 was dissolved in 10 ml of DMSO, 13 mmol of amine 2 and 13 mmol of K₂CO₃ were added and reaction mixture was stirred at 80° C. overnight. Then reaction mixture was diluted with 200 ml of water, precipitate formed, was filtered off and recrystalized from hexane.

Preparation of 4:

10 mmol of 3 was dissolved in 10 ml of water HBr and 10 mmol of Br₂ was added dropwice. Reaction mixture was stirred at room temperature overnight and then diluted with 200 ml of water and Na₂CO₃ was added carefully to ph 8 and than extracted with 50 ml of chloroform. Organic layer was separated, solvent was removed under reduced pressure and product was isolated by recrystallization from hexane.

Preparation of 6:

1 mol of 5 was suspended in 50 ml of 85% water formic acid and 80 ml of sulfuric acid were carefully added. Reaction mixture was stirred for 3 hours on boiling water bath and then cooled down. 800 ml of water were added and reaction mixture was alkalified by water ammonia solution to ph 3.5. Then reaction mixture was heated to 55° C. and 68.5 ml of Br₂ were added dropwise under liquid layer, keeping on temperature 55° C. and ph 3.5. Reaction mixture was stirred at this temperature overnight and after that it was cooled down and alkalified by water ammonia solution to ph 8. Precipitate was formed, filtered off, washed by water and dried.

Preparation of 8:

0.1 mol of 6 was dissolved in 200 ml of ethanol. 0.1 mol of 7 and 0.11 mol of triethylamine were added and reaction mixture was refluxed for 4 hours. Then solvent was removed under reduced pressure and residue was washed by water, filtered off and dried.

Preparation of 9:

0.2 mmol of 8 was dissolved in 2 ml of DMF and 0.2 mmol of 4 was added. Reaction mixture was stirred at 100° C. overnight. Then reaction mixture was diluted with 50 ml of water, extracted with 10 ml of chloroform, solvent was removed under reduced pressure and product was isolated by column chromatography (chloroform:methanol—20:1 as an eluent) or by HPLC.

Based on the above scheme, the following compounds were prepared.

| ID | structure | UPAC name | Identification |
|---|---|---|---|
| Z522- 0160 | 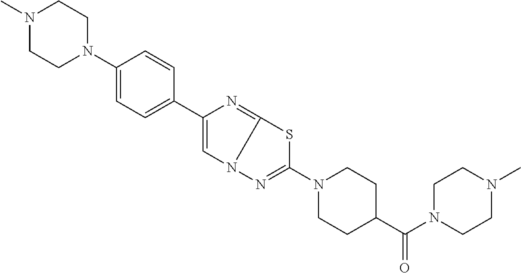 | 2-{4-[(4- methylpiperazin- 1-yl)carbonyl]- piperidin-1-yl}- 6-[4-(4-methyl- piperazin-1-yl)- phenyl]imidazo[2,1- b][1,3,4]- thiadiazole | LCMS: M + 1 = 509 |
| Z522- 0164 | 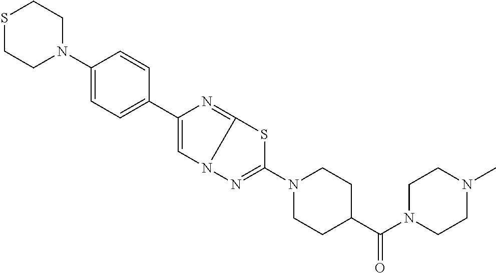 [SMILES: CN1CCN(C(=O)C2CCN(c3nn4cc(-c5ccc(N6CCSCC6)cc5)nc4s3)CC2)CC1] | 2-{4-[(4- methylpiperazin- 1-yl)carbonyl]- piperidin-1-yl}- 6-(4- thiomorpholin-4- ylphenyl)- imidazo[2,1-b]- [1,3,4]thiadiazole | LCMS: M + 1 = 512 |
| Z522- 0166 | 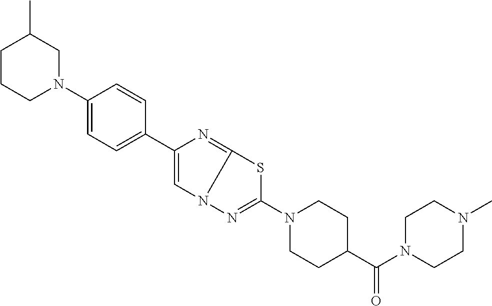 [SMILES: CC1CCCN(c2ccc(-c3cn4nc(N5CCC(C(=O)N6CCN(C)CC6)CC5)sc4n3)cc2)C1] | 2-{4-[(4- methylpiperazin- 1-yl)carbonyl]- piperidin-1-yl}- 6-[4-(3-methyl- piperidin-1-yl)- phenyl]imidazo[2,1- b][1,3,4]- thiadiazole | LCMS: M + 1 = 508 |
| Z522- 0167 | 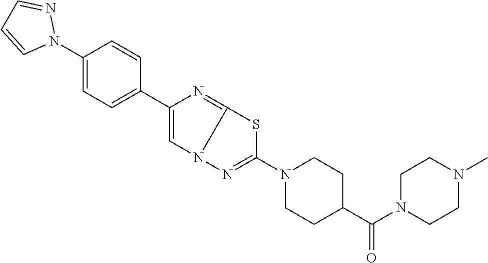 [SMILES: CN1CCN(C(=O)C2CCN(c3nn4cc(-c5ccc(-n6cccn6)cc5)nc4s3)CC2)CC1] | 2-{4-[(4-methyl- piperazin-1-yl)- carbonyl]piperidin- 1-yl}-6-[4- (1H-pyrazol-1- yl)- phenyl]imidazo[2,1- b][1,3,4]- thiadiazole | LCMS: M + 1 = 477 |
| Z522- 0170 | 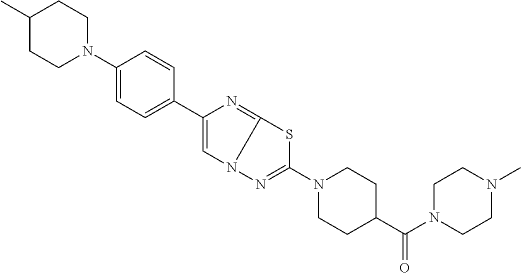 | 2-{4-[(4-methyl- piperazin-1- yl)carbonyl] piperidin-1-yl}-6-[4- (4-methylpiperidin- 1-yl)phenyl]- imidazo[2,1-b]- [1,3,4]thiadiazole | LCMS: M + 1 = 508 |
| Z522- 0174 | 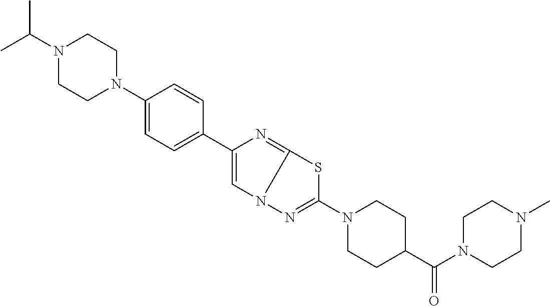 [SMILES: CC(C)N1CCN(c2ccc(-c3cn4nc(N5CCC(C(=O)N6CCN(C)CC6)CC5)sc4n3)cc2)CC1] | 6-[4-(4- isopropyl- piperazin-1-yl)- phenyl]-2-{4-[(4- methylpiperazin- 1-yl)carbonyl]- piperidin-1-yl}- imidazo[2,1-b]- [1,3,4]thiadiazole | LCMS: M + 1 = 537 |
| Z522- 0176 | 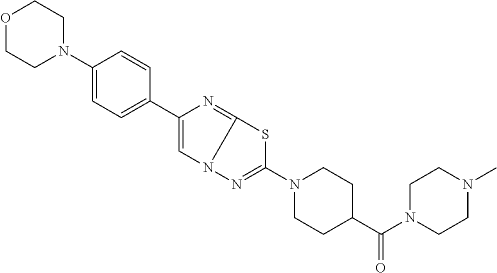 [SMILES: CN1CCN(C(=O)C2CCN(c3nn4cc(-c5ccc(N6CCOCC6)cc5)nc4s3)CC2)CC1] | 2-{4-[(4-methyl- piperazin-1-yl)- carbonyl]piperidin- 1-yl}-6-(4- morpholin-4-yl- phenyl)imidazo[2,1- b][1,3,4]- thiadiazole | LCMS: M + 1 = 496 |
| Z522- 0177 | 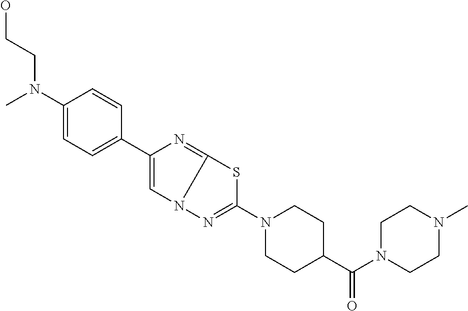 | 2-{methyl[4-(2- {4-[(4-methyl- piperazin-1-yl)- carbonyl]piperidin- 1-yl}imidazo- [2,1-b][1,3,4]- thiadiazol-6-yl)- phenyl]amino} ethanol | LCMS: M + 1 = 484 |
| Z522- 0179 | 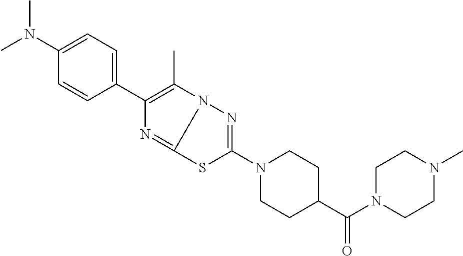 | N,N-dimethyl-4- (5-methyl-2-{4- [(4-methyl- piperazin-1- yl)carbonyl] piperidin-1- yl}imidazo-[2,1- b][1,3,4]- thiadiazol-6-yl)- aniline | LCMS: M + 1 = 468 |
| Z522- 0180 | 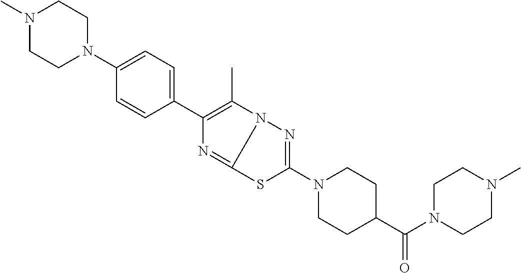 | 5-methyl-2-{4- [(4-methyl- piperazin-1- yl)carbonyl]piperidin- 1-yl}-6-[4-(4- methylpiperazin- 1-yl)phenyl]- imidazo[2,1-b]- [1,3,4]thiadiazole | LCMS: M + 1 = 523 |
| Z522- 0184 | 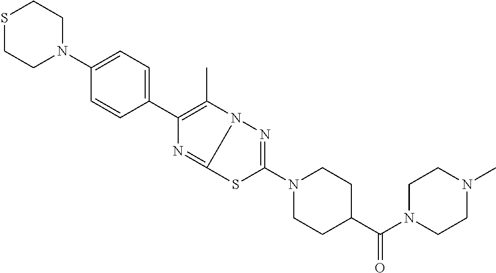 [SMILES: Cc1c(-c2ccc(N3CCSCC3)cc2)nc2sc(N3CCC(C(=O)N4CCN(C)CC4)CC3)nn12] | 5-methyl-2-{4- [(4-methyl- piperazin-1- yl)carbonyl] piperidin-1-yl}-6-(4- thiomorpholin-4- ylphenyl)imidazo [2,1-b][1,3,4]- thiadiazole | LCMS: M + 1 = 526 |
| Z522- 0185 | 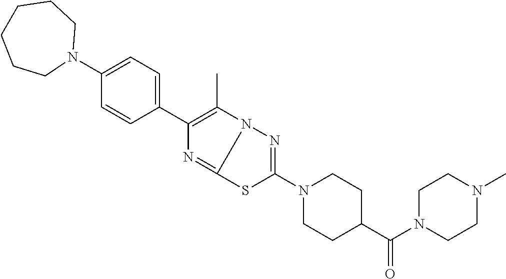 [SMILES: Cc1c(-c2ccc(N3CCCCCC3)cc2)nc2sc(N3CCC(C(=O)N4CCN(C)CC4)CC3)nn12] | 6-(4-azepan-1-yl- phenyl)-5- methyl-2-{4-[(4- methyl- piperazin-1- yl)carbonyl] piperidin-1- yl}imidazo-[2,1- b][1,3,4]- thiadiazole | LCMS: M + 1 = 522 |
| Z522- 0186 | 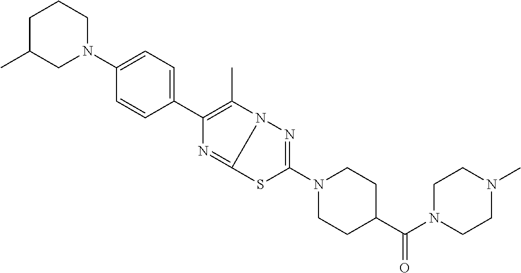 | 5-methyl-2-{4- [(4-methyl- piperazin-1- yl)carbonyl] piperidin-1-yl}-6-[4- (3-methylpiperidin- l-yl)phenyl]- imidazo[2,1-b]- [1,3,4]thiadiazole | LCMS: M + 1 = 522 |
| Z522- 0187 | 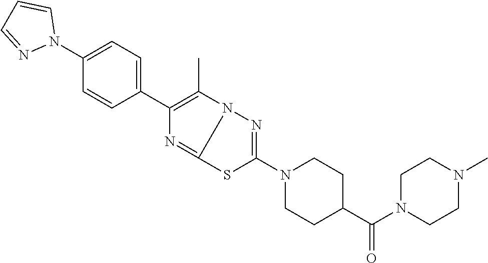 [SMILES: Cc1c(-c2ccc(-n3cccn3)cc2)nc2sc(N3CCC(C(=O)N4CCN(C)CC4)CC3)nn12] | 5-methyl-2-{4- [(4-methyl- piperazin-1-yl)- carbonyl]piperidin- 1-yl}-6-[4- (1H-pyrazol-1-yl)- phenyl]imidazo[2,1- b][1,3,4]- thiadiazole | LCMS: M + 1 = 491 |
| Z522- 0190 | 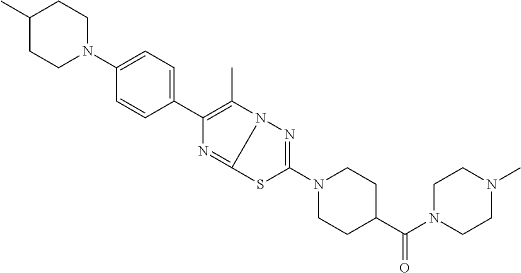 | 5-methyl-2-{4- [(4-methyl- piperazin-1-yl)- carbonyl]piperidin- 1-yl}-6-[4-(4- methylpiperidin- 1-yl)phenyl]- imidazo[2,1-b]- [1,3,4]thiadiazole | LCMS: M + 1 = 522 |
| Z522- 0191 | 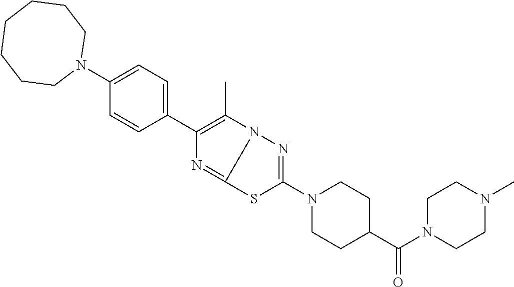 [SMILES: Cc1c(-c2ccc(N3CCCCCCC3)cc2)nc2sc(N3CCC(C(=O)N4CCN(C)CC4)CC3)nn12] | 6-(4-azocan-1- ylphenyl)-5- methyl-2-{4-[(4- methylpiperazin- 1-yl)carbonyl]- piperidin-1-yl}- imidazo[2,1-b]- [1,3,4]thiadiazole | LCMS: M + 1 = 536 |
| Z522- 0195 | 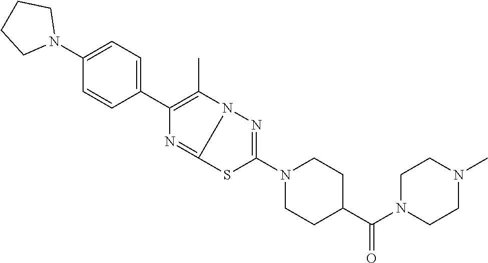 [SMILES: Cc1c(-c2ccc(N3CCCC3)cc2)nc2sc(N3CCC(C(=O)N4CCN(C)CC4)CC3)nn12] | 5-methyl-2-{4- [(4-methyl- piperazin-1- yl)carbonyl]piperidin- 1-yl}-6-(4- pyrrolidin-1- ylphenyl)imidazo [2,1-b][1,3,4]- thiadiazole | LCMS: M + 1 = 494 |
| Z522- 0196 | 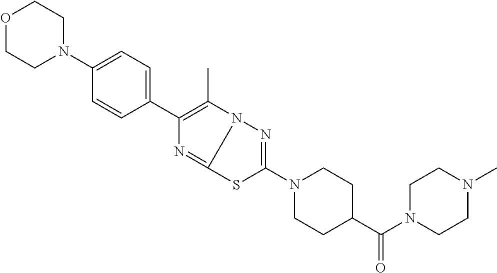 [SMILES: Cc1c(-c2ccc(N3CCOCC3)cc2)nc2sc(N3CCC(C(=O)N4CCN(C)CC4)CC3)nn12] | 5-methyl-2-{4- [(4-methyl- piperazin-1-yl)- carbonyl]piperidin- 1-yl}-6-(4- morpholin-4- ylphenyl)imidazo [2,1-b][1,3,4]- thiadiazole | LCMS: M + 1 = 510 |
| Z522- 0197 | 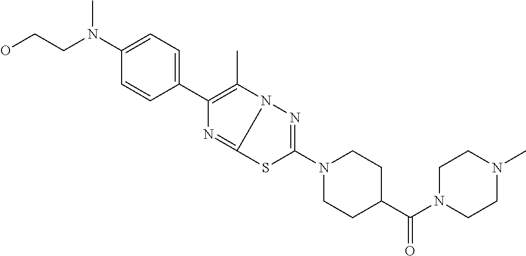 [SMILES: Cc1c(-c2ccc(N(C)CCO)cc2)nc2sc(N3CCC(C(=O)N4CCN(C)CC4)CC3)nn12] | 2-{methyl[4-(5- methyl-2-{4-[(4- methylpiperazin- 1-yl)carbonyl]- piperidin-1-yl}- imidazo[2,1-b]- [1,3,4]thiadiazol- 6-yl)phenyl]- amino}ethanol | LCMS: M + 1 = 498 |

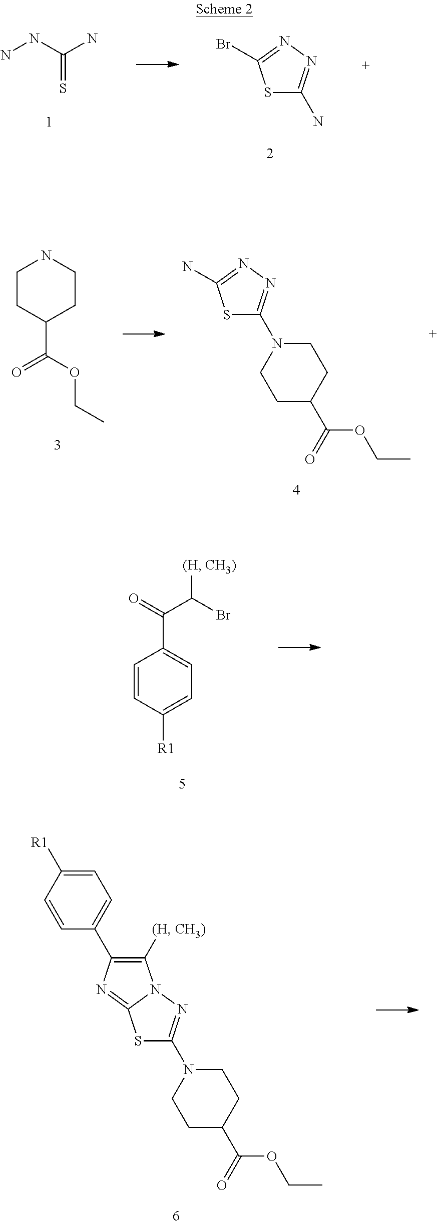

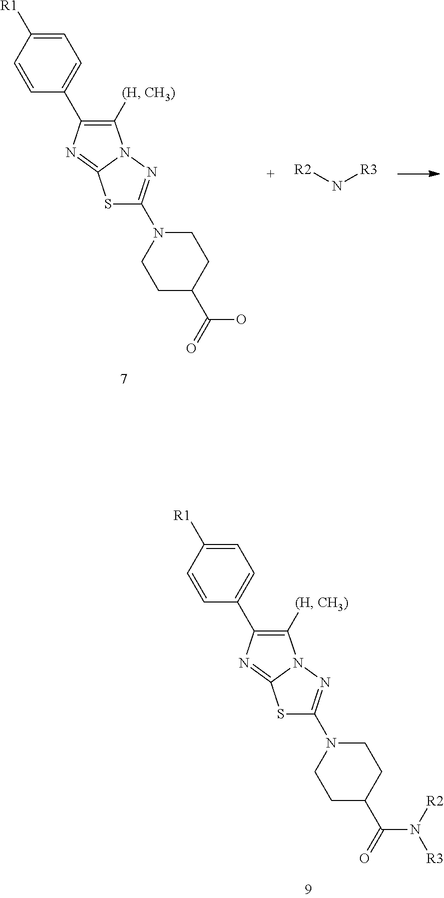

Preparation of 2:

1 mol of 1 was suspended in 50 ml of 85% water formic acid and 80 ml of sulfuric acid were carefully added. Reaction mixture was stirred for 3 hours on boiling water bath and then cooled down. 800 ml of water were added and reaction mixture was alkalified by water ammonia solution to ph 3.5. Then reaction mixture was heated to 55° C. and 68.5 ml of Br₂ were added dropwise under liquid layer, keeping on temperature 55° C. and ph 3.5. Reaction mixture was stirred at this temperature overnight and after that it was cooled down and alkalified by water ammonia solution to ph 8. Precipitate was formed, filtered off, washed by water and dried.

Preparation of 4:

0.1 mol of 2 was dissolved in 200 ml of ethanol. 0.1 mol of 3 and 0.11 mol of triethylamine were added and reaction mixture was refluxed for 4 hours. Then solvent was removed under reduced pressure and residue was washed by water, filtered off and dried.

Preparation of 6:

0.1 mol of 4 was dissolved in 100 ml of DMF and 0.1 mol of 5 was added. Reaction mixture was stirred at 100° C. overnight. Then solvent was removed under reduced pressure and residue was diluted with 250 ml of water, extracted with 200 ml of chloroform, solvent was removed under reduced pressure and product was isolated by column chromatography (chloroform:methanol—20:1 as an eluent).

Preparation of 7:

0.1 mol of 6 was dissolved in 200 ml of ethanol and solution of 4.8 g of NaOH in 50 ml of water was added. Reaction mixture was refluxed for 3 hours. All solvents were removed under reduced pressure, then 200 ml of water was added and acidified with hydrochloric acid. Precipitate was filtered off and washed with water.

Preparation of 9:

0.3 mmol of 7 was stirred with 0.33 mmol of CDI in 2 ml of DMF at 100° C. for 1.5 hour. Then amine 8 (0.33) was added and reaction mixture was stirred at 100° C. overnight. Then reaction mixture was cooled down, diluter with 100 ml of water. Precipitate was filtered off and purified by recristallization from ethanol or by column chromatography (chloroform:methanol—20:1 as an eluent). Based on the above scheme, the following compounds were prepared.

| ID | structure | UPAC name | Identification |
|---|---|---|---|
| Z522- 0199 | 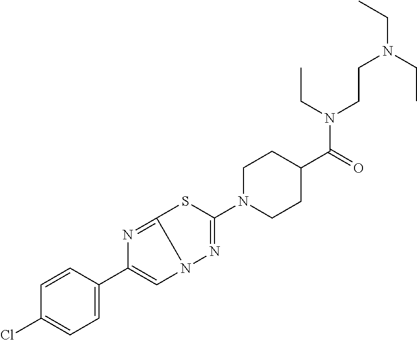 [SMILES: CCN(CC)CCN(CC)C(=O)C1CCN(c2nn3cc(-c4ccc(Cl)cc4)nc3s2)CC1] | 1-[6-(4-chlorophenyl) imidazo[2,1- b][1,3,4]thiadiazol- 2-yl]-N-[2- (diethylamino)ethyl]- N-ethylpiperidine-4- carboxamide | LCMS: M + 1 = 490 |
| Z522- 0200 | 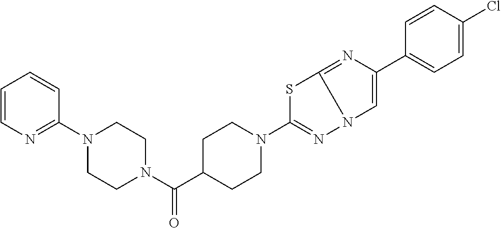 [SMILES: O=C(C1CCN(c2nn3cc(-c4ccc(Cl)cc4)nc3s2)CC1)N1CCN(c2ccccn2)CC1] | 6-(4-chlorophenyl)-2- {4-[(4-pyridin- 2-ylpiperazin-1- yl)carbonyl]piperidin- 1-yl}imidazo[2,1- b][1,3,4]thiadiazole | LCMS: M + 1 = 509 |
| Z522- 0202 | 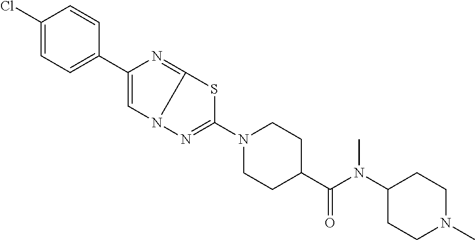 [SMILES: CN1CCC(N(C)C(=O)C2CCN(c3nn4cc(-c5ccc(Cl)cc5)nc4s3)CC2)CC1] | 1-[6-(4-chlorophenyl) imidazo[2,1- b][1,3,4]thiadiazol-2- yl]-N-methyl- N-(1-methylpiperidin- 4-yl)piperidine-4- carboxamide | LCMS: M + 1 = 474 |
| Z522- 0203 | 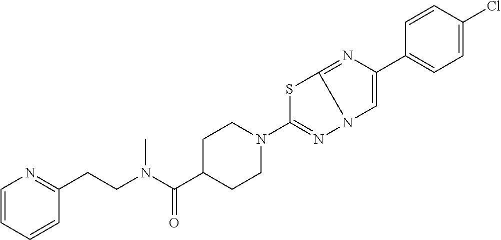 [SMILES: CN(CCc1ccccn1)C(=O)C1CCN(c2nn3cc(-c4ccc(Cl)cc4)nc3s2)CC1] | 1-[6-(4-chlorophenyl) imidazo[2,1- b][1,3,4]thiadiazol-2- yl]-N-methyl- N-(2-pyridin-2-ylethyl) piperidine-4- carboxamide | LCMS: M + 1 = 482 |
| Z522- 0204 | 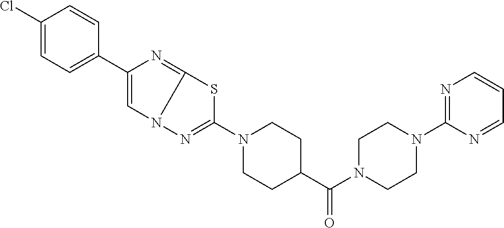 [SMILES: O=C(C1CCN(c2nn3cc(-c4ccc(Cl)cc4)nc3s2)CC1)N1CCN(c2ncccn2)CC1] | 6-(4-chlorophenyl)-2- {4-[(4-pyrimidin- 2-ylpiperazin-1- yl)carbonyl]piperidin- 1-yl}imidazo[2,1- b][1,3,4]thiadiazole | LCMS: M + 1 = 510 |
| Z522- 0205 | 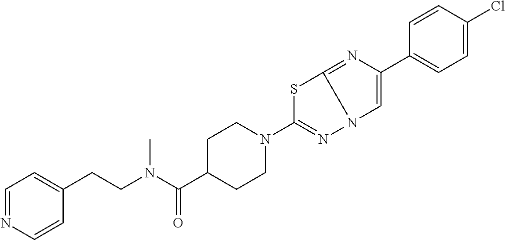 [SMILES: CN(CCc1ccncc1)C(=O)C1CCN(c2nn3cc(-c4ccc(Cl)cc4)nc3s2)CC1] | 1-[6-(4-chlorophenyl) imidazo[2,1- b][1,3,4]thiadiazol-2- yl]-N-methyl- N-(2-pyridin-4-ylethyl) piperidine-4- carboxamide | LCMS: M + 1 = 482 |
| Z522- 0206 | 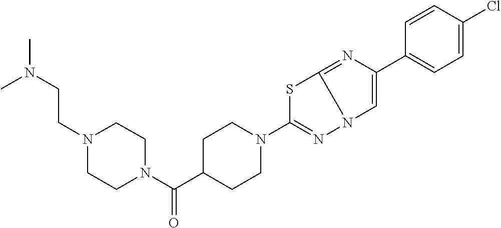 [SMILES: CN(C)CCN1CCN(C(=O)C2CCN(c3nn4cc(-c5ccc(Cl)cc5)nc4s3)CC2)CC1] | 1-[6-(4-chlorophenyl) imidazo[2,1- b][1,3,4]thiadiazol-2- yl]-N-methyl- N-(2-pyridin-4-ylethyl) piperidine-4- carboxamide | LCMS: M + 1 = 503 |
| Z522- 0207 | 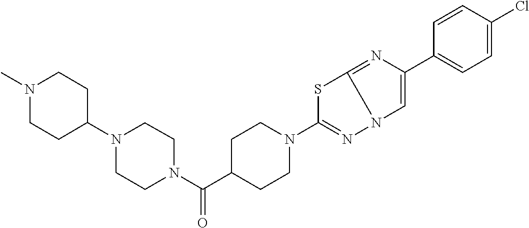 [SMILES: CN1CCC(N2CCN(C(=O)C3CCN(c4nn5cc(-c6ccc(Cl)cc6)nc5s4)CC3)CC2)CC1] | 6-(4-chlorophenyl)- 2-(4-{[4-(1- methylpiperidin-4-yl) piperazin-1- yl]carbonyl}piperidin- 1-yl)imidazo[2,1- b][1,3,4]thiadiazole | LCMS: M + 1 = 530 |
| Z522- 0209 | 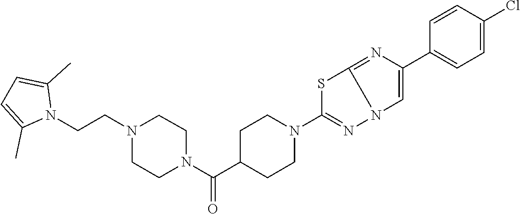 [SMILES: Cc1ccc(C)n1CCN1CCN(C(=O)C2CCN(c3nn4cc(-c5ccc(Cl)cc5)nc4s3)CC2)CC1] | 6-(4-chlorophenyl)-2- [4-({4-[2-(2,5- dimethyl-1H-pyrrol-1- yl)ethyl]piperazin-1- yl}carbonyl)piperidin- 1-yl]imidazo[2,1- b][1,3,4]thiadiazole | LCMS: M + 1 = 553 |
| Z522- 0211 | 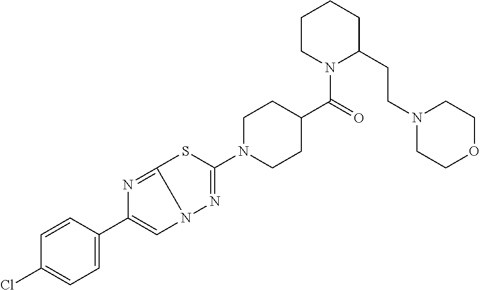 [SMILES: O=C(C1CCN(c2nn3cc(-c4ccc(Cl)cc4)nc3s2)CC1)N1CCCCC1CCN1CCOCC1] | 6-(4-chlorophenyl)- 2-(4-{[2-(2- morpholin-4-ylethyl) piperidin-1- yl]carbonyl}piperidin- 1-yl)imidazo[2,1- b][1,3,4]thiadiazole | LCMS: M + 1 = 544 |
| Z522- 0212 | 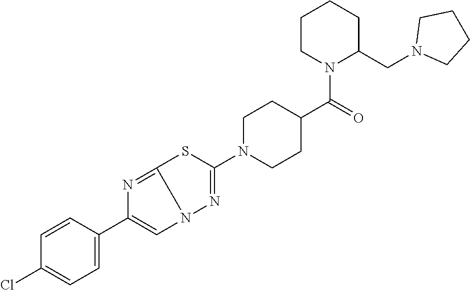 [SMILES: O=C(C1CCN(c2nn3cc(-c4ccc(Cl)cc4)nc3s2)CC1)N1CCCCC1CN1CCCC1] | 6-(4-chlorophenyl)- 2-(4-{[2- (pyrrolidin-1-ylmethyl) piperidin-1- yl]carbonyl}piperidin- 1-yl)imidazo[2,1- b][1,3,4]thiadiazole | LCMS: M + 1 = 514 |
| Z522- 0213 | 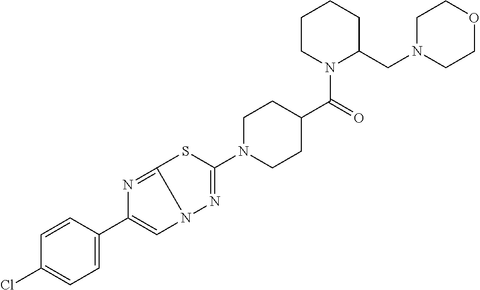 [SMILES: O=C(C1CCN(c2nn3cc(-c4ccc(Cl)cc4)nc3s2)CC1)N1CCCCC1CN1CCOCC1] | 6-(4-chlorophenyl)- 2-(4-{[2- (morpholin-4-ylmethyl) piperidin-1- yl]carbonyl}piperidin-1- yl)imidazo[2,1-b][1,3,4] thiadiazole | LCMS: M + 1 = 530 |
| Z522- 0214 | 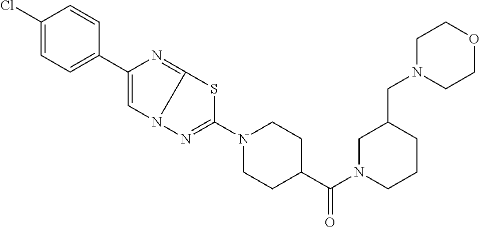 [SMILES: O=C(C1CCN(c2nn3cc(-c4ccc(Cl)cc4)nc3s2)CC1)N1CCCC(CN2CCOCC2)C1] | 6-(4-chlorophenyl)- 2-(4-{[3- (morpholin-4-ylmethyl) piperidin-1- yl]carbonyl}piperidin- 1-yl)imidazo[2,1- b][1,3,4]thiadiazole | LCMS: M + 1 = 530 |
| Z522- 0215 | 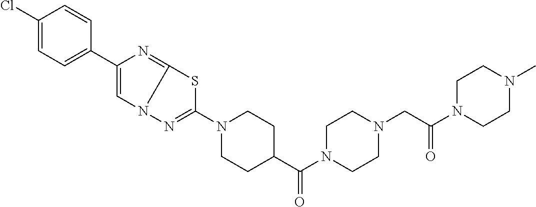 [SMILES: CN1CCN(C(=O)CN2CCN(C(=O)C3CCN(c4nn5cc(-c6ccc(Cl)cc6)nc5s4)CC3)CC2)CC1] | 6-(4-chlorophenyl)-2- [4-({4-[2-(4- methylpiperazin-1-yl)-2- oxoethyl]piperazin-1- yl}carbonyl)piperidin-1- yl]imidazo[2,1- b][1,3,4]thiadiazole | LCMS: M + 1 = 572 |
| Z522- 0216 | 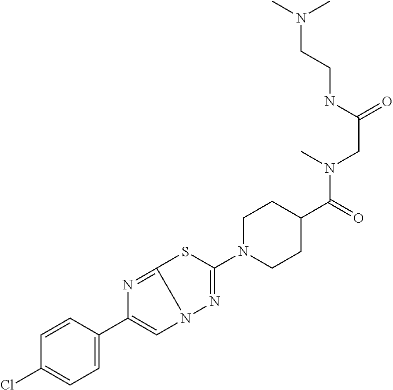 [SMILES: CN(C)CCNC(=O)CN(C)C(=O)C1CCN(c2nn3cc(-c4ccc(Cl)cc4)nc3s2)CC1] | 1-[6-(4-chlorophenyl) imidazo[2,1- b][1,3,4]thiadiazol- 2-yl]-N⁴-(2-[2- (dimethylamino) ethyl]amino-2- oxoethyl)-N⁴-methyl-4- piperidinecarboxamide | LCMS: M + 1 = 505 |
| Z522- 0218 | 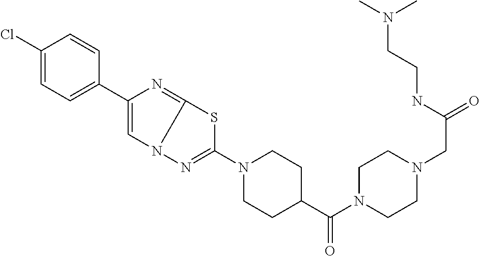 [SMILES: CN(C)CCNC(=O)CN1CCN(C(=O)C2CCN(c3nn4cc(-c5ccc(Cl)cc5)nc4s3)CC2)CC1] | 2-[4-({1-[6-(4- chlorophenyl) imidazo[2,1- b][1,3,4]thiadiazol-2- yl]piperidin-4- yl}carbonyl)piperazin- 1-yl]-N-[2- (dimethylamino)ethyl] acetamide | LCMS: M + 1 = 560 |
| Z522- 0219 | 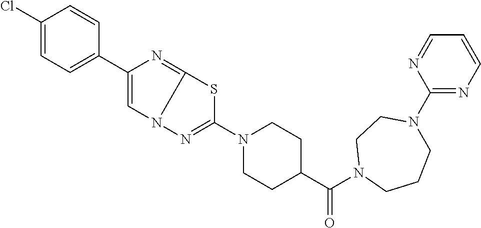 [SMILES: O=C(C1CCN(c2nn3cc(-c4ccc(Cl)cc4)nc3s2)CC1)N1CCCN(c2ncccn2)CC1] | 6-(4-chlorophenyl)- 2-{4-[(4- pyrimidin-2-yl-1,4- diazepan-1- yl)carbonyl]piperidin- 1-yl}imidazo[2,1- b][1,3,4]thiadiazole | LCMS: M + 1 = 524 |
| Z522- 0220 | 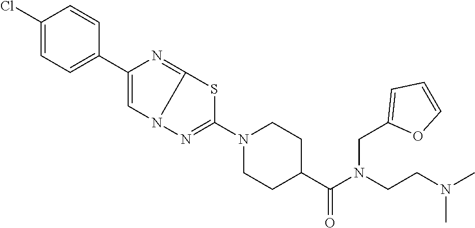 [SMILES: CN(C)CCN(Cc1ccco1)C(=O)C1CCN(c2nn3cc(-c4ccc(Cl)cc4)nc3s2)CC1] | 1-[6-(4-chlorophenyl) imidazo[2,1- b][1,3,4]thiadiazol-2- yl]-N-[2- (dimethylamino) ethyl]-N-(2- furylmethyl)piperidine- 4-carboxamide | LCMS: M + 1 = 514 |
| Z522- 0221 | 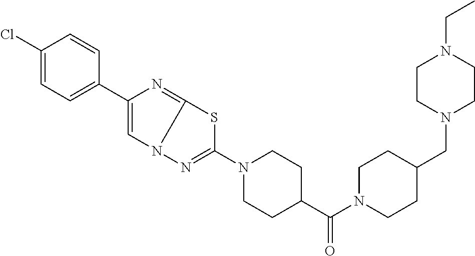 [SMILES: CCN1CCN(CC2CCN(C(=O)C3CCN(c4nn5cc(-c6ccc(Cl)cc6)nc5s4)CC3)CC2)CC1] | 6-(4-chlorophenyl)- 2-[4-({4-[(4- ethylpiperazin-1- yl)methyl]piperidin-1- yl}carbonyl)piperidin- 1-yl]imidazo[2,1- b][1,3,4]thiadiazole | LCMS: M + 1 = 557 |
| Z522- 0222 | 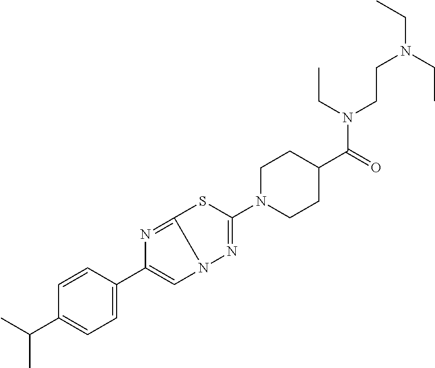 [SMILES: CCN(CC)CCN(CC)C(=O)C1CCN(c2nn3cc(-c4ccc(C(C)C)cc4)nc3s2)CC1] | N-[2-(diethylamino) ethyl]-N-ethyl-1- [6-(4-isopropylphenyl) imidazo[2,1- b][1,3,4]thiadiazol-2- yl]piperidine-4- carboxamide | LCMS: M + 1 = 497 |
| Z522- 0223 | 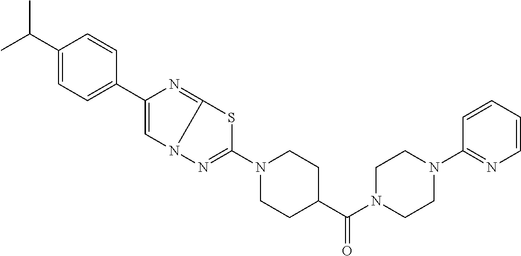 [SMILES: CC(C)c1ccc(-c2cn3nc(N4CCC(C(=O)N5CCN(c6ccccn6)CC5)CC4)sc3n2)cc1] | 6-(4-isopropyl- phenyl)-2-{4-[(4- pyridin-2-ylpiperazin- 1-yl)carbonyl] piperidin-1- yl}imidazo[2,1- b][1,3,4]thiadiazole | LCMS: M + 1 = 516 |
| Z522- 0224 | 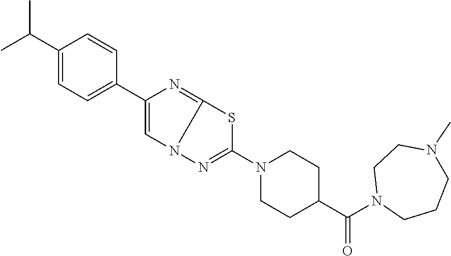 [SMILES: CC(C)c1ccc(-c2cn3nc(N4CCC(C(=O)N5CCCN(C)CC5)CC4)sc3n2)cc1] | 6-(4-isopropylphenyl)- 2-{4-[(4- methyl-1,4-diazepan- 1-yl)carbonyl] piperidin-1- yl}imidazo[2,1- b][1,3,4]thiadiazole | LCMS: M + 1 = 467 |
| Z522- 0225 | 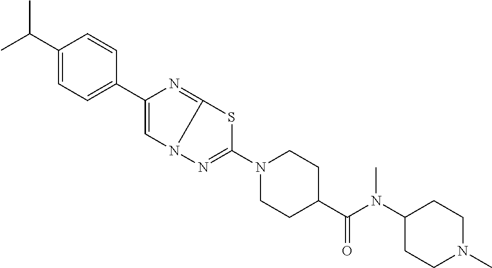 [SMILES: CC(C)c1ccc(-c2cn3nc(N4CCC(C(=O)N(C)C5CCN(C)CC5)CC4)sc3n2)cc1] | 1-[6-(4- isopropylphenyl) imidazo[2,1- b][1,3,4]thiadiazol- 2-yl]-N-methyl- N-(1-methylpiperidin- 4-yl)piperidine-4- carboxamide | LCMS: M + 1 = 481 |
| Z522- 0226 | 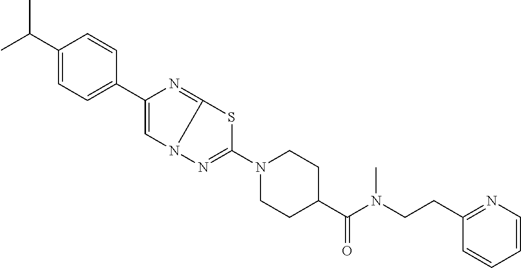 [SMILES: CC(C)c1ccc(-c2cn3nc(N4CCC(C(=O)N(C)CCc5ccccn5)CC4)sc3n2)cc1] | 1-[6-(4- isopropylphenyl) imidazo[2,1- b][1,3,4]thiadiazol- 2-yl]-N-methyl- N-(2-pyridin-2- ylethyl)piperidine-4- carboxamide | LCMS: M + 1 = 489 |
| Z522- 0227 | 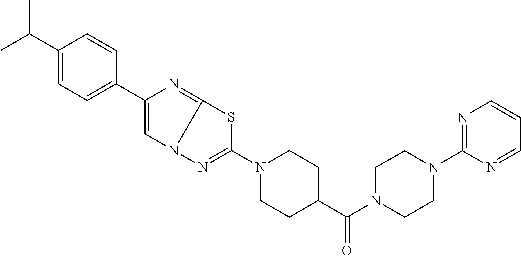 [SMILES: CC(C)c1ccc(-c2cn3nc(N4CCC(C(=O)N5CCN(c6ncccn6)CC5)CC4)sc3n2)cc1] | 6-(4-isopropyl- phenyl)-2-{4-[(4- pyrimidin-2-ylpiperazin- 1-yl)carbonyl]piperidin- 1-yl}imidazo[2,1- b][1,3,4]thiadiazole | LCMS: M + 1 = 517 |
| Z522- 0228 | 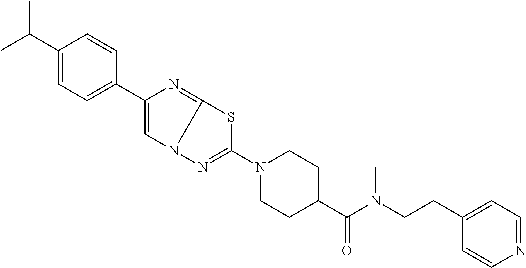 [SMILES: CC(C)c1ccc(-c2cn3nc(N4CCC(C(=O)N(C)CCc5ccncc5)CC4)sc3n2)cc1] | 1-[6-(4- isopropylphenyl) imidazo[2,1- b][1,3,4]thiadiazol-2- yl]-N-methyl- N-(2-pyridin-4- ylethyl)piperidine-4- carboxamide | LCMS: M + 1 = 489 |
| Z522- 0229 | 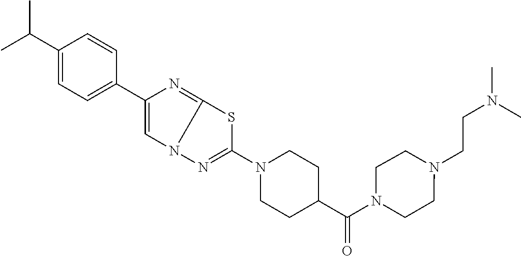 [SMILES: CC(C)c1ccc(-c2cn3nc(N4CCC(C(=O)N5CCN(CCN(C)C)CC5)CC4)sc3n2)cc1] | {2-[4-({1-[6-(4- isopropylphenyl) imidazo[2,1- b][1,3,4]thiadiazol-2- yl]piperidin-4- yl}carbonyl)piperazin- 1-yl]ethyl} dimethylamine | LCMS: M + 1 = 510 |
| Z522- 0230 | 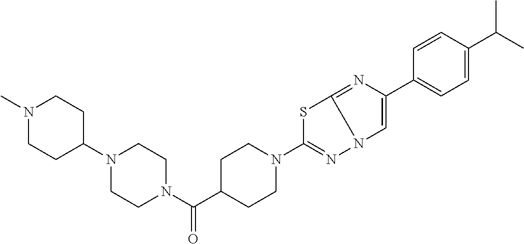 [SMILES: CC(C)c1ccc(-c2cn3nc(N4CCC(C(=O)N5CCN(C6CCN(C)CC6)CC5)CC4)sc3n2)cc1] | 6-(4-isopropylphenyl)- 2-(4-{[4-(1- methylpiperidin-4- yl)piperazin-1- yl]carbonyl}piperidin- 1-yl)imidazo[2,1- b][1,3,4]thiadiazole | LCMS: M + 1 = 536 |
| Z522- 0232 | 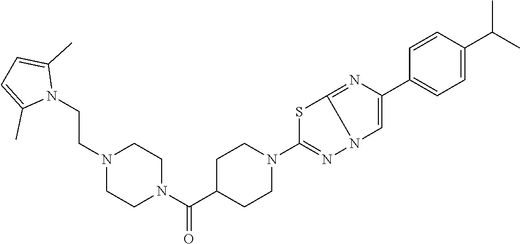 [SMILES: Cc1ccc(C)n1CCN1CCN(C(=O)C2CCN(c3nn4cc(-c5ccc(C(C)C)cc5)nc4s3)CC2)CC1] | 2-[4-({4-[2-(2,5- dimethyl-1H- pyrrol-1-yl)ethyl] piperazin-1- yl}carbonyl)piperidin- 1-yl]-6-(4- isopropylphenyl) imidazo[2,1- b][1,3,4]thiadiazole | LCMS: M + 1 = 560 |
| Z522- 0233 | 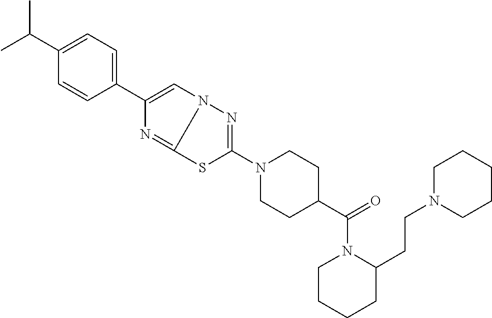 [SMILES: CC(C)c1ccc(-c2cn3nc(N4CCC(C(=O)N5CCCCC5CCN5CCCCC5)CC4)sc3n2)cc1] | 6-(4-isopropylphenyl)- 2-(4-{[2-(2- piperidin-1-ylethyl) piperidin-1- yl]carbonyl}piperidin- 1-yl)imidazo[2,1- b][1,3,4]thiadiazole | LCMS: M + 1 = 549 |
| Z522- 0234 | 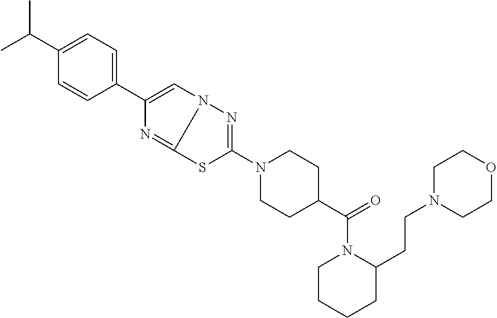 [SMILES: CC(C)c1ccc(-c2cn3nc(N4CCC(C(=O)N5CCCCC5CCN5CCOCC5)CC4)sc3n2)cc1] | 6-(4-isopropylphenyl)- 2-(4-{[2-(2- morpholin-4-ylethyl) piperidin-1- yl]carbonyl}piperidin- 1-yl)imidazo[2,1- b][1,3,4]thiadiazole | LCMS: M + 1 = 551 |
| Z522- 0235 | 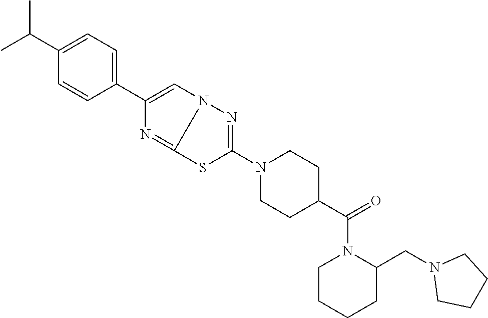 [SMILES: CC(C)c1ccc(-c2cn3nc(N4CCC(C(=O)N5CCCCC5CN5CCCC5)CC4)sc3n2)cc1] | 6-(4-isopropylphenyl)- 2-(4-{[2- (pyrrolidin-1-ylmethyl) piperidin-1- yl]carbonyl}piperidin-1- yl)imidazo[2,1- b][1,3,4]thiadiazole | LCMS: M + 1 = 521 |
| Z522- 0237 | 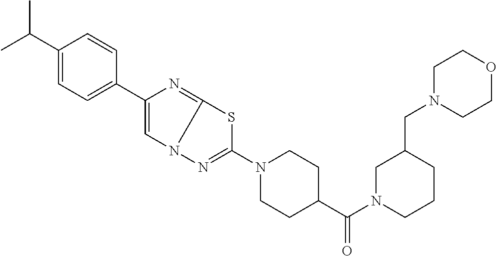 [SMILES: CC(C)c1ccc(-c2cn3nc(N4CCC(C(=O)N5CCCC(CN6CCOCC6)C5)CC4)sc3n2)cc1] | 6-(4-isopropyl- phenyl)-2-(4-{[3- (morpholin-4-yl- methyl)piperidin-1- yl]carbonyl}piperidin- 1-yl)imidazo[2,1- b][1,3,4]thiadiazole | LCMS: M + 1 = 537 |
| Z522- 0238 | 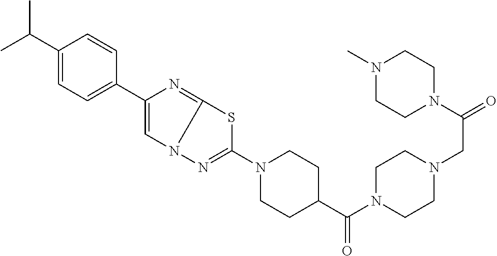 [SMILES: CC(C)c1ccc(-c2cn3nc(N4CCC(C(=O)N5CCN(CC(=O)N6CCN(C)CC6)CC5)CC4)sc3n2)cc1] | 6-(4-isopropylphenyl)- 2-[4-({4-[2- (4-methylpiperazin- 1-yl)-2- oxoethyl]piperazin- 1-yl}carbonyl) piperidin-1- yl]imidazo[2,1- b][1,3,4]thiadiazole | LCMS: M + 1 = 579 |
| Z522- 0239 | 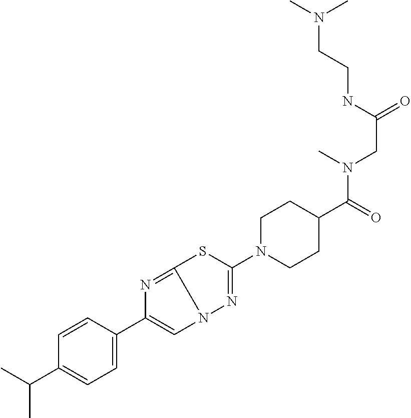 [SMILES: CC(C)c1ccc(-c2cn3nc(N4CCC(C(=O)N(C)CC(=O)NCCN(C)C)CC4)sc3n2)cc1] | N⁴-(2-[2- (dimethylamino) ethyl]amino-2- oxoethyl)-1-[6-(4- isopropylphenyl) imidazo[2,1- b][1,3,4]thiadiazol- 2-yl]-N⁴-methyl- 4-piperidinecarboxamide | LCMS: M + 1 = 512 |
| Z522- 0240 | 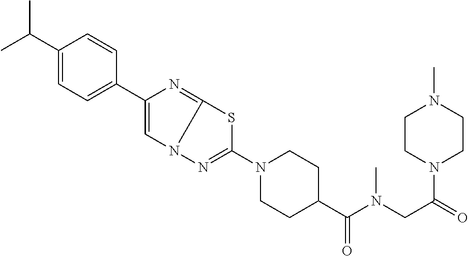 [SMILES: CC(C)c1ccc(-c2cn3nc(N4CCC(C(=O)N(C)CC(=O)N5CCN(C)CC5)CC4)sc3n2)cc1] | 1-[6-(4- isopropylphenyl) imidazo[2,1- b][1,3,4]thiadiazol- 2-yl]-N⁴-methyl- N⁴-[2-(4-methyl- piperazino)-2- oxoethyl]-4- piperidinecarboxamide | LCMS: M + 1 = 524 |
| Z522- 0241 | 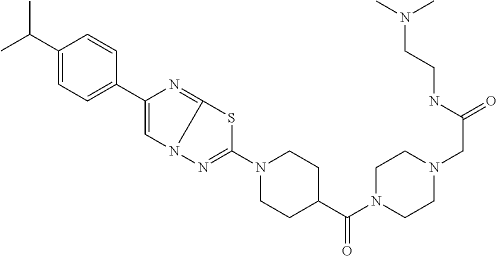 [SMILES: CC(C)c1ccc(-c2cn3nc(N4CCC(C(=O)N5CCN(CC(=O)NCCN(C)C)CC5)CC4)sc3n2)cc1] | N-[2-(dimethylamino) ethyl]-2-[4- ({1-[6-(4- isopropylphenyl) imidazo[2,1- b][1,3,4]thiadiazol- 2-yl]piperidin-4- yl}carbonyl)piperazin- 1-yl]acetamide | LCMS: M + 1 = 567 |
| Z522- 0242 | 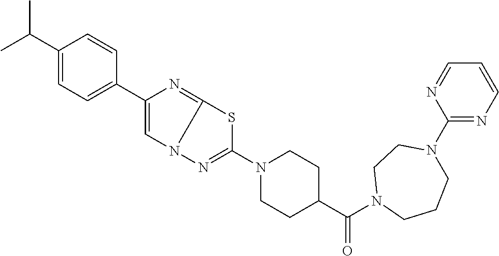 [SMILES: CC(C)c1ccc(-c2cn3nc(N4CCC(C(=O)N5CCCN(c6ncccn6)CC5)CC4)sc3n2)cc1] | 6-(4-isopropylphenyl)- 2-{4-[(4- pyrimidin-2-yl- 1,4-diazepan-1- yl)carbonyl]piperidin- 1-yl}imidazo[2,1- b][1,3,4]thiadiazole | LCMS: M + 1 = 531 |
| Z522- 0244 | 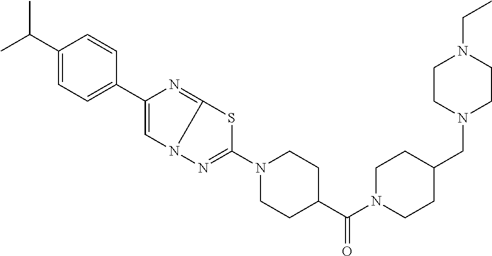 [SMILES: CCN1CCN(CC2CCN(C(=O)C3CCN(c4nn5cc(-c6ccc(C(C)C)cc6)nc5s4)CC3)CC2)CC1] | 2-[4-({4-[(4- ethylpiperazin-1- yl)methyl]piperidin- 1-yl}carbonyl) piperidin-1-yl]-6-(4- isopropylphenyl) imidazo[2,1- b][1,3,4]thiadiazole | LCMS: M + 1 = 564 |

### Pharmaceutical Compositions

A pharmaceutical composition, according to the present invention can include an effective amount of one or more compounds of formula (I) and/or (II) and a pharmaceutically acceptable carrier, diluent, excipient or combination thereof.

The term “pharmaceutical composition” refers to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids such as defined above. Pharmaceutical compositions will generally be tailored to the specific intended route of administration.

The term “physiologically acceptable” defines a carrier, diluent or excipient that does not abrogate the biological activity and properties of the compound.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

When administered orally, one or more compounds described herein (e.g., a compound of Formula (I) and/or Formula (II), or a pharmaceutically acceptable salt thereof) can be formulated as tablets, pills, dragees, capsules, liquids, gels, syrups, slurries, suspensions and the like, for oral ingestion by a subject to be treated. Injectables can be prepared in conventional forms, either as liquid solutions or suspensions, solid forms suitable for solution or suspension in liquid prior to injection, or as emulsions. Pharmaceutical compositions for intranasal delivery may also include drops and sprays often prepared to assist in simulating nasal secretions.

The effect of the compounds Z522-XXX on the growth profile of cancer cells which express Fer was tested. The cells that were tested were: the colon cancer cell lines HT29 and HCT 116, RKO. The effect of each of the compounds on FS cells, a non malingnant fibroblastic cell line, was also studied. The results are provided in Table 1 below.

Generally, cells were seeded in 96 wells microplates and were left to grow untreated overnight. Each of the compounds was dissolved in DMSO and was then added to each well in a final concentration of 5 μM. The concentration of DMSO in each well was 0.4% v/v. Untreated cells and cells subjected to 0.4% DMSO alone, served as controls. The percentage of viable cells in each well was determined 96 hours after compounds addition, using the XTT test (Berridge, M. V. et al, 2005).

TABLE 1

| | 5 μM 96 h | | | | | | | |
|---|---|---|---|---|---|---|---|---|
| Treatment | HCT116 | HCT116 | HT29 | HT29 | RKO | RKO | FS | FS |
| Control | 100% | 100% | 100% | 100% | 100% | 100% | 100% | 100% |
| DMSO | | | | | | | | |
| Z522-0160 | 105% | 108% | 117% | 91% | 92% | 109% | 101% | 104% |
| Z522-0164 | 80% | 97% | 50% | 42% | 60% | 63% | 104% | 95% |
| Z522-0166 | 30% | 23% | 4% | 4% | 6% | 6% | 101% | 92% |
| Z522-0167 | 94% | 83% | 87% | 86% | 36% | 47% | 96% | 92% |
| Z522-0170 | 2% | 3% | 5% | 2% | 3% | 4% | 90% | 68% |
| Z522-0174 | 105% | 124% | 77% | 78% | 93% | 97% | 117% | 113% |
| Z522-0176 | 109% | 116% | 112% | 112% | 92% | 89% | 103% | 100% |
| Z522-0177 | 107% | 104% | 116% | 121% | 97% | 96% | 120% | 116% |
| Z522-0179 | 101% | 108% | 103% | 100% | 109% | 103% | 91% | 117% |
| Z522-0180 | 94% | 105% | 89% | 90% | 95% | 103% | 101% | 101% |
| Z522-0184 | 77% | 87% | 50% | 44% | 41% | 53% | 71% | 86% |
| Z522-0185 | 40% | 42% | 19% | 11% | 14% | 15% | 92% | 94% |
| Z522-0186 | 23% | 26% | 7% | 7% | 8% | 11% | 82% | 94% |
| Z522-0187 | 110% | 97% | 99% | 111% | 56% | 56% | 92% | 94% |
| Z522-0190 | 45% | 51% | 7% | 6% | 9% | 9% | 86% | 101% |
| Z522-0191 | 39% | 41% | 12% | 10% | 23% | 35% | 103% | 101% |
| Z522-0195 | 111% | 113% | 91% | 82% | 80% | 75% | 109% | 119% |
| Z522-0196 | 105% | 104% | 104% | 101% | 88% | 93% | 86% | 94% |
| Z522-0197 | 95% | 108% | 102% | 110% | 111% | 117% | 113% | 104% |
| Z522-0199 | −3% | −1% | −1% | 1% | −3% | −3% | 19% | 21% |
| Z522-0200 | 94% | 88% | 92% | 98% | 81% | 78% | 95% | 103% |
| Z522-0202 | 5% | −1% | 1% | 0% | −3% | −1% | 73% | 69% |
| Z522-0203 | 75% | 75% | 74% | 88% | 48% | 48% | 90% | 94% |
| Z522-0204 | 97% | 110% | 105% | 97% | 92% | 76% | 96% | 93% |
| Z522-0205 | 78% | 71% | 88% | 85% | 52% | 62% | 88% | 111% |
| Z522-0206 | 1% | −1% | −2% | −3% | −4% | −3% | 2% | 6% |
| Z522-0207 | 0% | 4% | 3% | 0% | −4% | −3% | 3% | 16% |
| Z522-0209 | 71% | 59% | 25% | 30% | 52% | 55% | 89% | 96% |
| Z522-0211 | 72% | 61% | 64% | 60% | −2% | −2% | 75% | 81% |
| Z522-0212 | 2% | −1% | 1% | 0% | −4% | −3% | −3% | 1% |
| Z522-0213 | 84% | 88% | 87% | 99% | 18% | 13% | 87% | 98% |
| Z522-0214 | 70% | 71% | 53% | 57% | 24% | 23% | 104% | 90% |
| Z522-0215 | 63% | 58% | 14% | 11% | 6% | 10% | 85% | 109% |
| Z522-0216 | 68% | 86% | 10% | 11% | 0% | 2% | 86% | 93% |
| Z522-0218 | 51% | 19% | 3% | 4% | −3% | −1% | 82% | 84% |
| Z522-0219 | 75% | 67% | 91% | 95% | 48% | 45% | 89% | 94% |
| Z522-0220 | 1% | 3% | 0% | −3% | −8% | −6% | 61% | 49% |
| Z522-0221 | 1% | 1% | −3% | −3% | −5% | −4% | −9% | −5% |
| Z522-0222 | 1% | −2% | −1% | −1% | −6% | −5% | 56% | 56% |
| Z522-0223 | 75% | 80% | 83% | 89% | 64% | 81% | 87% | 92% |
| Z522-0224 | 0% | −1% | 0% | 0% | −1% | −1% | 60% | 67% |
| Z522-0225 | 1% | 0% | −1% | −3% | −6% | −2% | 65% | 28% |
| Z522-0226 | 80% | 80% | 101% | 92% | 72% | 64% | 91% | 99% |
| Z522-0227 | 72% | 84% | 91% | 91% | 63% | 63% | 89% | 91% |
| Z522-0228 | 82% | 70% | 58% | 61% | 51% | 55% | 111% | 113% |
| Z522-0229 | 87% | 99% | 74% | 77% | 45% | 47% | 79% | 101% |
| Z522-0230 | 2% | 0% | −2% | −2% | −3% | −4% | 22% | 39% |
| Z522-0232 | 79% | 68% | 20% | 22% | 35% | 38% | 106% | 93% |
| Z522-0233 | 1% | −1% | 1% | 0% | −3% | 0% | −7% | 3% |
| Z522-0234 | 60% | 54% | 12% | 7% | −1% | −1% | 62% | 105% |
| Z522-0235 | 1% | 1% | −2% | −1% | −6% | −4% | −5% | 14% |
| Z522-0237 | 74% | 89% | 13% | 8% | 11% | 8% | 82% | 105% |
| Z522-0238 | 54% | 49% | 6% | 7% | 1% | 3% | 85% | 99% |
| Z522-0239 | 80% | 45% | 3% | 2% | 1% | 1% | 83% | 96% |
| Z522-0240 | 82% | 68% | 50% | 53% | 19% | 15% | 80% | 101% |
| Z522-0241 | 33% | 36% | 0% | 0% | −2% | 0% | 78% | 88% |
| Z522-0242 | 88% | 86% | 72% | 66% | 55% | 50% | 89% | 123% |
| Z522-0244 | −1% | 0% | 1% | 0% | −2% | 0% | −5% | −3% |

1. A pharmaceutical composition comprising a compound of formulae (I) or (II) or a pharmaceutically acceptable salt thereof: 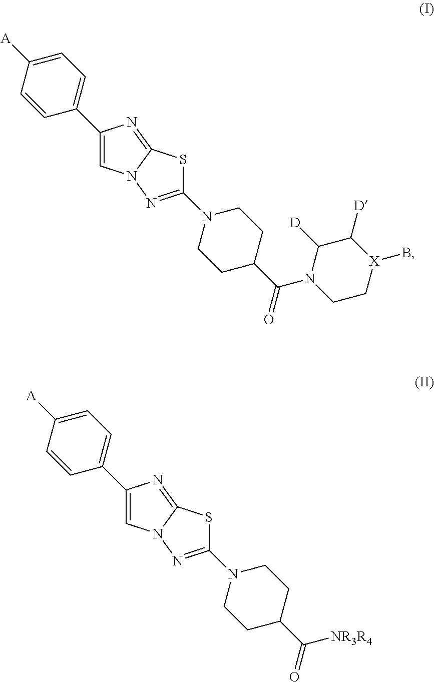
wherein:
X is N or C;
A is: H, halogen, a straight or branched C₁-C₅ alkyl, NR₁R₂, or a C₅₋₈-heterocyclyl or heteroaryl having 1, 2 or 3 heteroatoms selected from N, O, S, that may be substituted by a straight or branched C₁-C₅ alkyl;
B is: H, straight or branched C₁-C₅, (CH₂)ₙ—NR₁R₂, (CH₂)ₙ—C(═O)—NR₁R₂(CH₂)ₙ—NR₁R₂, a C₅₋₇-heterocyclyl or heteroaryl having 1, 2 or 3 heteroatoms selected from N, O, S, that may be substituted by straight or branched C₁-C₅ alkyl, (CH₂)ₙ—C₅₋₇-heterocyclyl or heteroaryl having 1, 2 or 3 heteroatoms selected from N, O, S, that may be substituted by straight or branched C₁-C₅ alkyl, or (CH₂)ₙ—C(═O)—C₅₋₇-hetercyclyl or heteroaryl, having 1, 2 or 3 heteroatoms selected from N, O, S, that may be substituted by straight or branched C₁-C₅ alkyl;
D and D′ are independently H or (CH₂)ₙ—C₅₋₇-heterocyclyl or -heteroaryl, having 1, 2 or 3 heteroatoms selected from N, O, S;
provided that, when X is C, one of D and D′ is H and the other is (CH₂)ₙ-heterocylcyl and B is H; and, when X is N, D and D′ are both H;
R₁ and R₂ are independently H, straight or branched C₁-C₅ alkyl, or straight or branched C₁-C₅—OH;
R₃ is H, straight or branched C₁-C₅ alkyl, or (CH₂)ₙNR₁R₂;
R₄ is (CH₂)ₙ—C(═O)—NR₁R₂—NR₁R₂, or (CH₂)ₙ— C₅₋₇-hetercyclyl or heteroaryl having 1, 2 or 3 heteroatoms selected from N, O, S; and
n is 0, 1, 2 or 3;
provided that the following compounds: 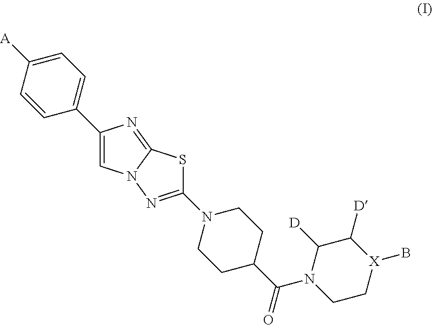
where:
A is H, halogen, or a straight or branched C₁-C₅ alkyl;
D and D′ are H; and
B is H or straight or branched C₁-C₅ are excluded.

2. The pharmaceutical composition of claim 1, wherein, in formula (I):
X is N;
A is: halogen, a straight or branched C₁-C₅ alkyl, NR₁R₂, or a C₅₋₈-heterocyclyl or heteroaryl having 1, 2 or 3 heteroatoms selected from N, O, S, that may be substituted by a straight or branched C₁-C₅ alkyl;
B is: straight or branched C₁-C₅, (CH₂)ₙ—NR₁R₂, (CH₂)ₙ—C(═O)⁻NR₁R₂(CH₂)ₙ—NR₁R₂, a C₅₋₇-heterocyclyl or heteroaryl having 1, 2 or 3 heteroatoms selected from N, O, S, that may be substituted by straight or branched C₁-C₅ alkyl; (CH₂)ₙ—C₅₋₇-hetercyclyl or heteroaryl, or (CH₂)ₙ—C(═O)—C₅₋₇-heterocyclyl or heteroaryl.

3. The pharmaceutical composition of claim 1, wherein:
A is: halogen, a straight or branched C₁-C₅ alkyl, NR₁R₂, or a C₅₋₈-heterocyclyl or heteroaryl having 1 or 2 heteroatoms selected from N, O, S, that may be substituted by a straight or branched C₁-C₅ alkyl; and
B is: straight or branched C₁-C₅, (CH₂)ₙ—NR₁R₂, (CH₂)ₙ—C(═O)—NR₁R₂(CH₂)ₙ—NR₁R₂, a C₅₋₇-heterocyclyl or heteroaryl having 1 or 2 heteroatoms selected from N, O, S, that may be substituted by straight or branched C₁-C₅ alkyl, (CH₂)ₙ—C₅₋₇-hetercyclyl or heteroaryl, or (CH₂)ₙ—C(═O)— C₅₋₇-heterocyclyl or heteroaryl having 1 or 2 heteroatoms selected from N, O, S that may be substituted by straight or branched C₁-C₅ alkyl.

4. The pharmaceutical composition of claim 1, wherein the C₅₋₈-heterocyclyl or heteroaryl is selected from the group consisting of pyrrolidine, pyrazole, piperidine, piperazine, morpholine, thiomorpholine, azepane, and azocane, and the C₅₋₇-hetercyclyl or heteroaryl is selected from the group consisting of pyridine, pyrimidine, piperidine, pyrazole, pyrrolidine, and morpholine.

5. The pharmaceutical composition of claim 1, wherein, in formula (I):
X is C;
A is: halogen, a straight or branched C₁-C₅ alkyl, NR₁R₂, or a C₅₋₈-heterocyclyl or heteroaryl having 1, 2 or 3 heteroatoms selected from N, O, S, that may be substituted by a straight or branched C₁-C₅ alkyl; and
D and D′ are independently H or (CH₂)ₙ—C₅₋₇-heterocyclyl or heteroaryl, having 1, 2 or 3 heteroatoms selected from N, O, S

6. The pharmaceutical composition according to claim 5, wherein the C₅₋₈-heterocyclyl or heteroaryl is selected from the group consisting of pyrrolidine, pyrazole, piperidine, piperazine, morpholine, thiomorpholine, azepane, and azocane, and the C₅₋₇-hetercyclyl or heteroaryl is selected from the group consisting of pyridine, pyrimidine, piperidine, pyrazole, pyrrolidine, and morpholine.

7. The pharmaceutical composition according to claim 1, selected from the group consisting of:

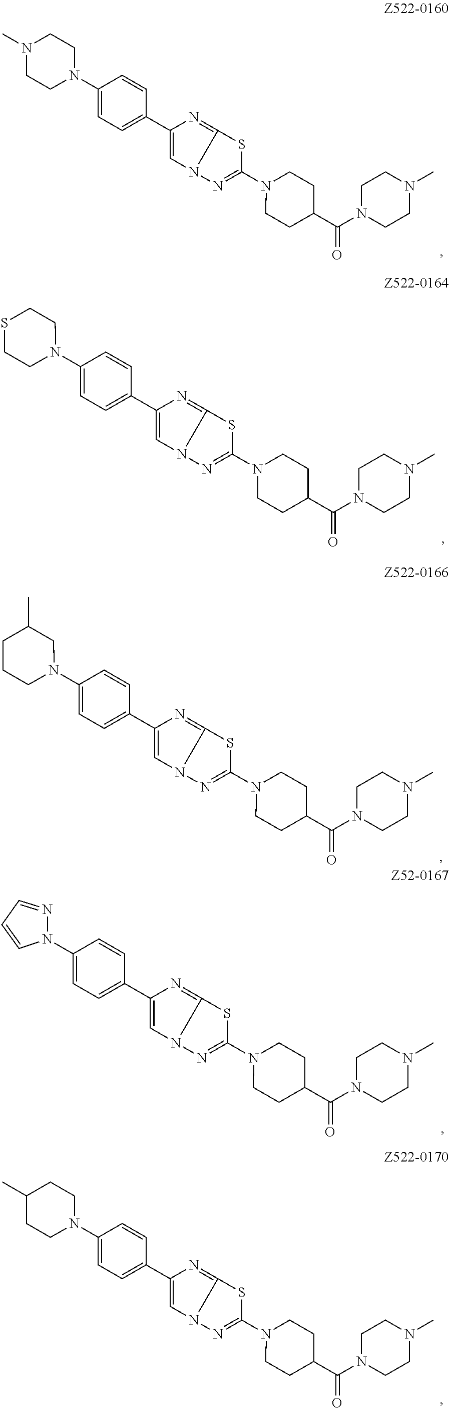

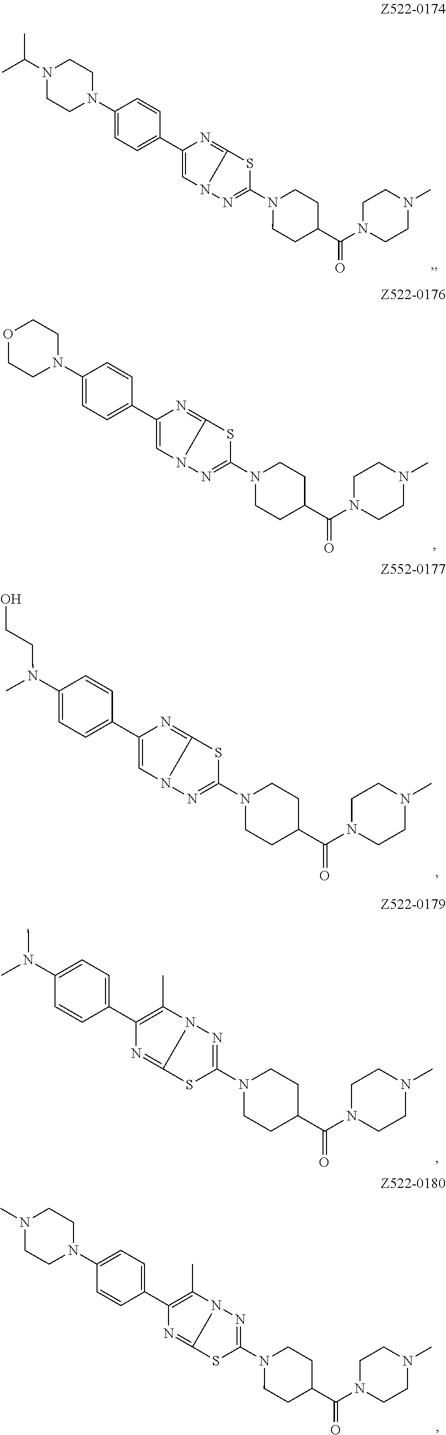

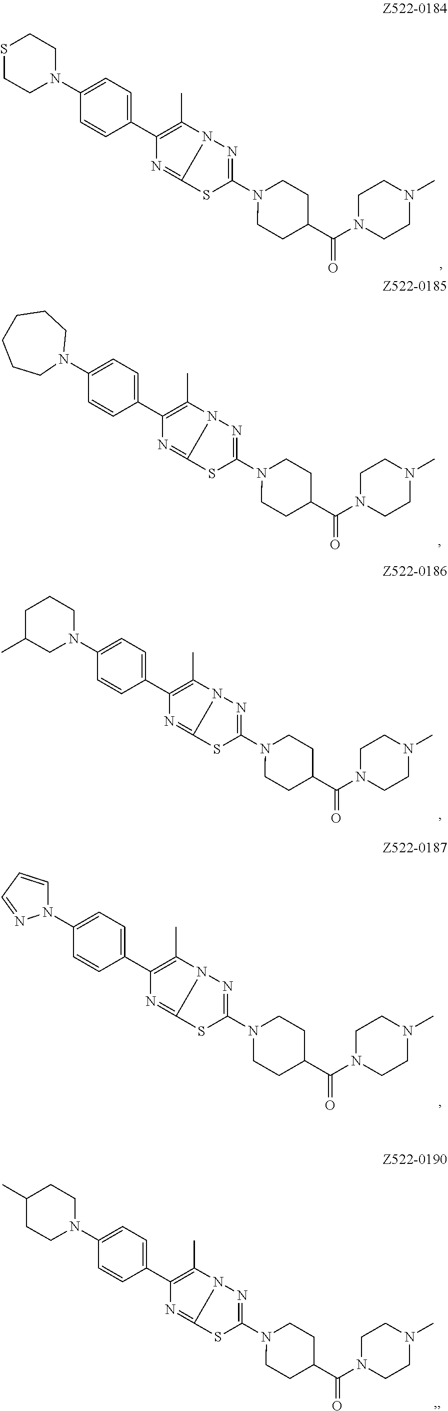

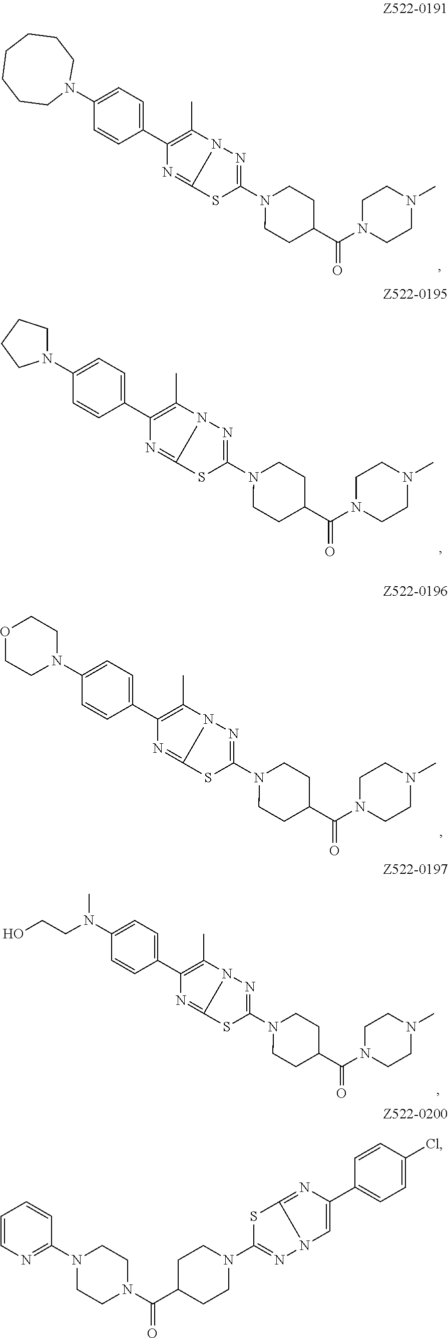 [SMILES: Cc1c(-c2ccc(N3CCCCCCC3)cc2)nc2sc(N3CCC(C(=O)N4CCN(C)CC4)CC3)nn12 ; Cc1c(-c2ccc(N3CCCC3)cc2)nc2sc(N3CCC(C(=O)N4CCN(C)CC4)CC3)nn12 ; Cc1c(-c2ccc(N3CCOCC3)cc2)nc2sc(N3CCC(C(=O)N4CCN(C)CC4)CC3)nn12 ; Cc1c(-c2ccc(N(C)CCO)cc2)nc2sc(N3CCC(C(=O)N4CCN(C)CC4)CC3)nn12 ; O=C(C1CCN(c2nn3cc(-c4ccc(Cl)cc4)nc3s2)CC1)N1CCN(c2ccccn2)CC1]

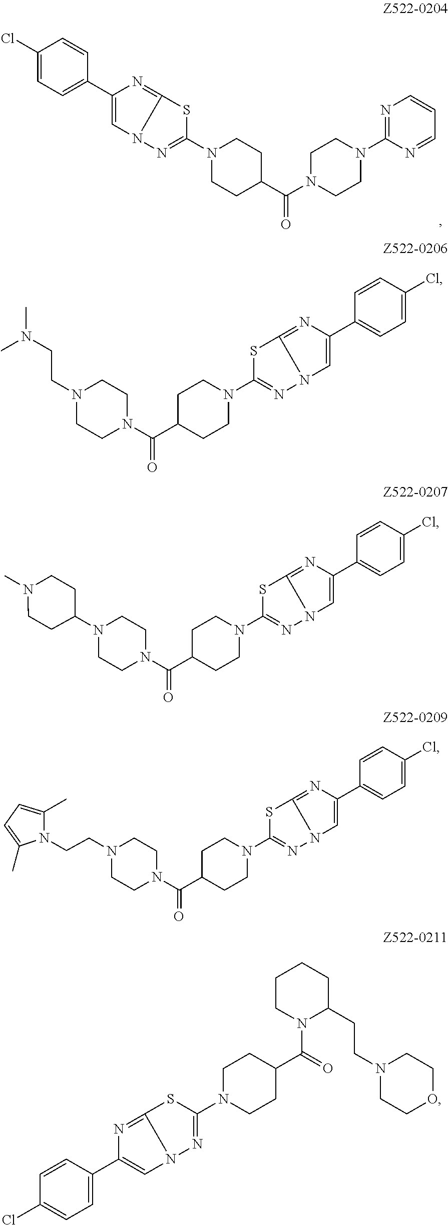 [SMILES: O=C(C1CCN(c2nn3cc(-c4ccc(Cl)cc4)nc3s2)CC1)N1CCN(c2ncccn2)CC1 ; CN(C)CCN1CCN(C(=O)C2CCN(c3nn4cc(-c5ccc(Cl)cc5)nc4s3)CC2)CC1 ; CN1CCC(N2CCN(C(=O)C3CCN(c4nn5cc(-c6ccc(Cl)cc6)nc5s4)CC3)CC2)CC1 ; Cc1ccc(C)n1CCN1CCN(C(=O)C2CCN(c3nn4cc(-c5ccc(Cl)cc5)nc4s3)CC2)CC1 ; O=C(C1CCN(c2nn3cc(-c4ccc(Cl)cc4)nc3s2)CC1)N1CCCCC1CCN1CCOCC1]

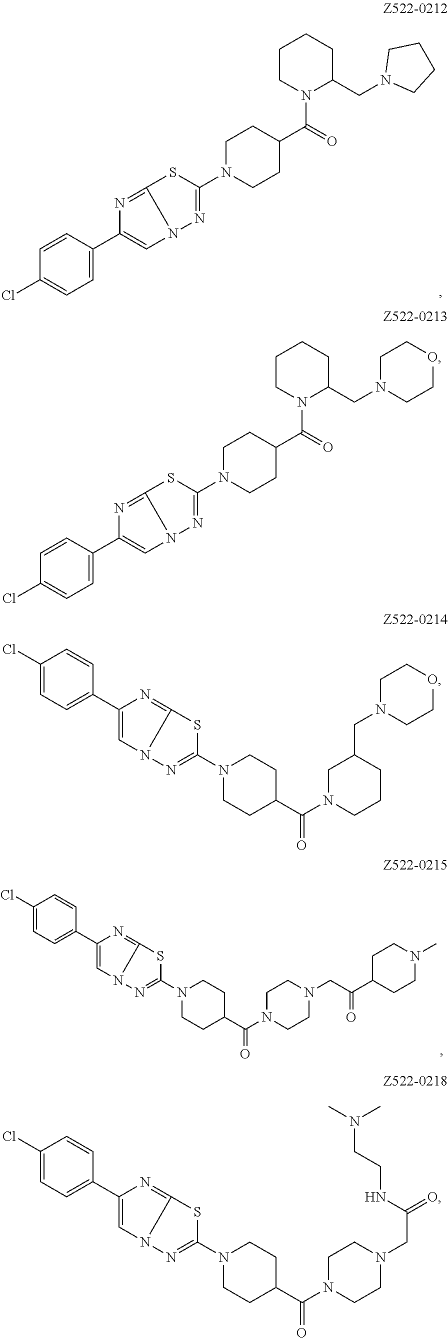 [SMILES: O=C(C1CCN(c2nn3cc(-c4ccc(Cl)cc4)nc3s2)CC1)N1CCCCC1CN1CCCC1 ; O=C(C1CCN(c2nn3cc(-c4ccc(Cl)cc4)nc3s2)CC1)N1CCCCC1CN1CCOCC1 ; O=C(C1CCN(c2nn3cc(-c4ccc(Cl)cc4)nc3s2)CC1)N1CCCC(CN2CCOCC2)C1 ; CN1CCC(C(=O)CN2CCN(C(=O)C3CCN(c4nn5cc(-c6ccc(Cl)cc6)nc5s4)CC3)CC2)CC1 ; CN(C)CCNC(=O)CN1CCN(C(=O)C2CCN(c3nn4cc(-c5ccc(Cl)cc5)nc4s3)CC2)CC1]

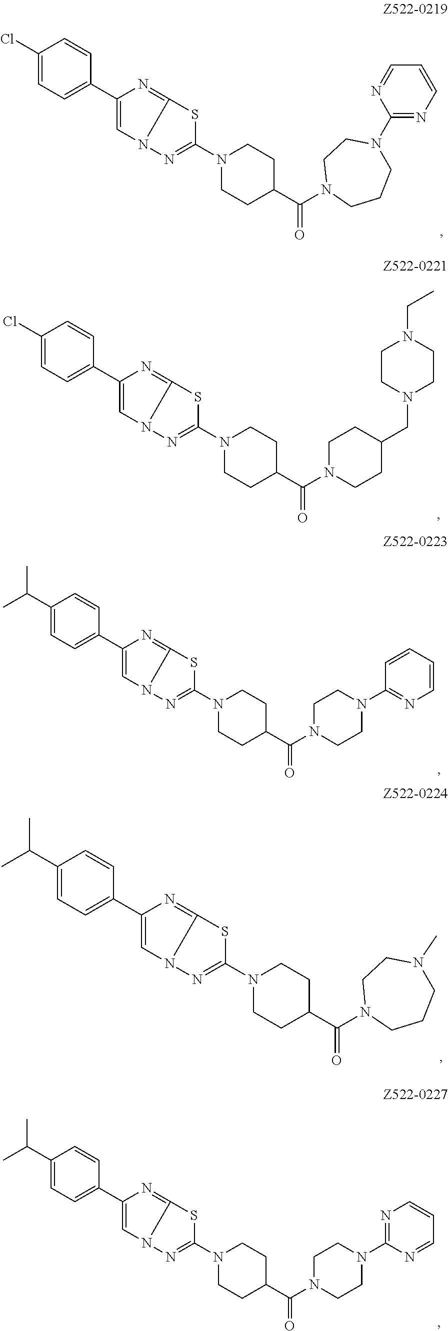

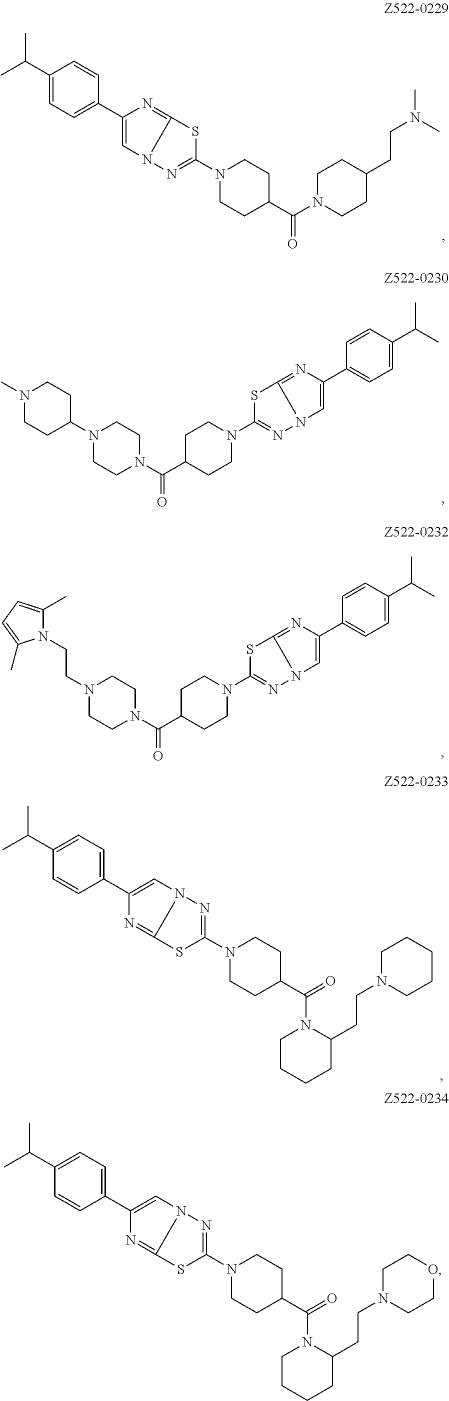

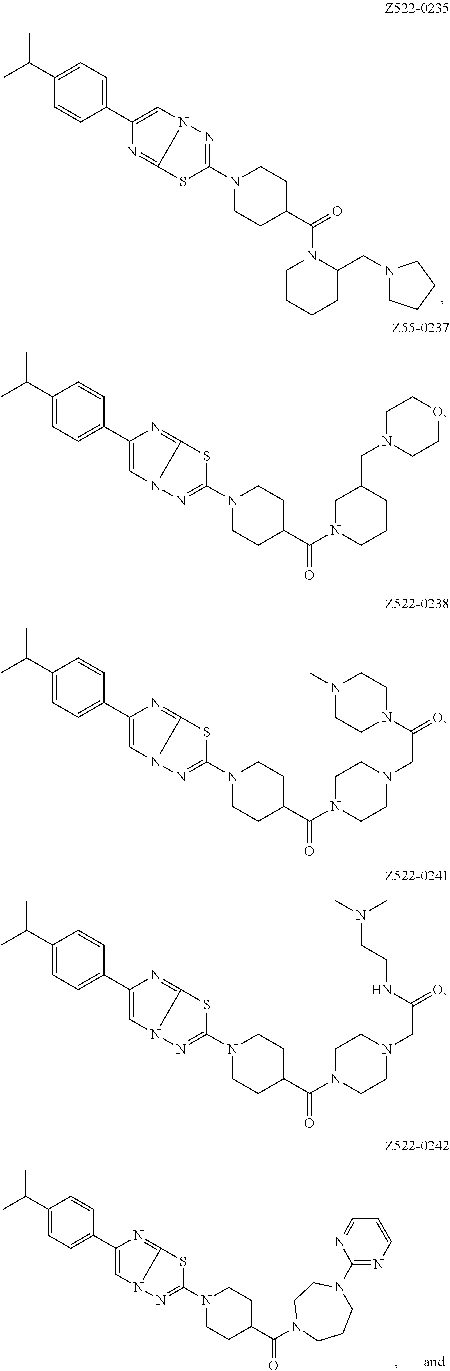 [SMILES: CC(C)c1ccc(-c2cn3nc(N4CCC(C(=O)N5CCCCC5CN5CCCC5)CC4)sc3n2)cc1 ; CC(C)c1ccc(-c2cn3nc(N4CCC(C(=O)N5CCCC(CN6CCOCC6)C5)CC4)sc3n2)cc1 ; CC(C)c1ccc(-c2cn3nc(N4CCC(C(=O)N5CCN(CC(=O)N6CCN(C)CC6)CC5)CC4)sc3n2)cc1 ; CC(C)c1ccc(-c2cn3nc(N4CCC(C(=O)N5CCN(CC(=O)NCCN(C)C)CC5)CC4)sc3n2)cc1 ; CC(C)c1ccc(-c2cn3nc(N4CCC(C(=O)N5CCCN(c6ncccn6)CC5)CC4)sc3n2)cc1]

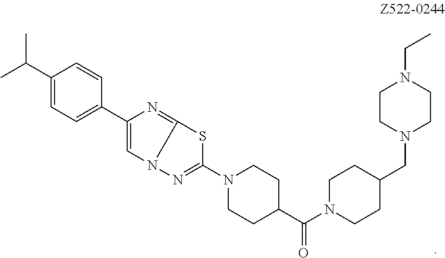 [SMILES: CCN1CCN(CC2CCN(C(=O)C3CCN(c4nn5cc(-c6ccc(C(C)C)cc6)nc5s4)CC3)CC2)CC1]

8. The pharmaceutical composition according to claim 1, selected from the group consisting of:

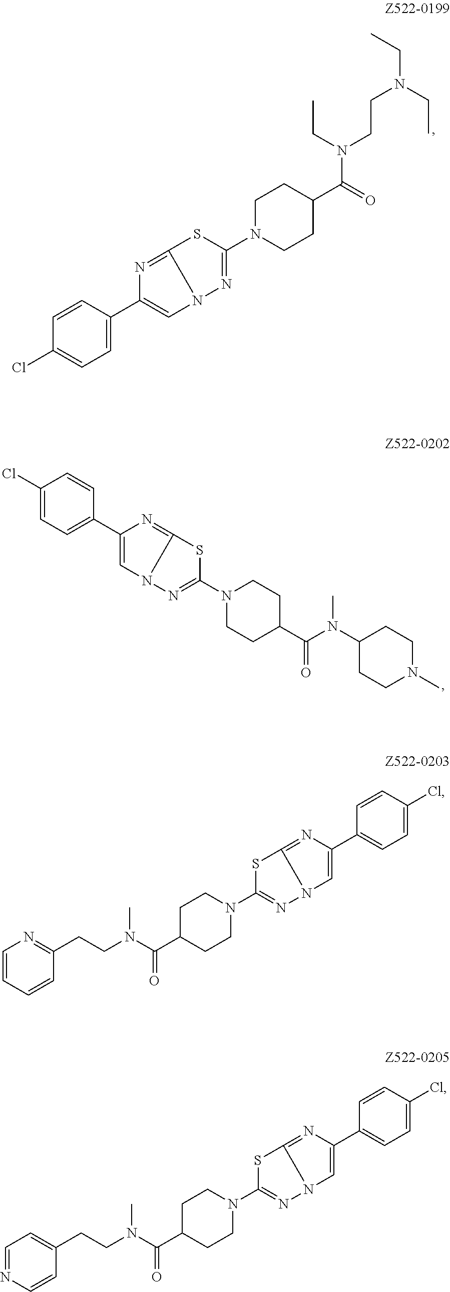 [SMILES: CCN(CC)CCN(CC)C(=O)C1CCN(c2nn3cc(-c4ccc(Cl)cc4)nc3s2)CC1 ; CN1CCC(N(C)C(=O)C2CCN(c3nn4cc(-c5ccc(Cl)cc5)nc4s3)CC2)CC1 ; CN(CCc1ccccn1)C(=O)C1CCN(c2nn3cc(-c4ccc(Cl)cc4)nc3s2)CC1 ; CN(CCc1ccncc1)C(=O)C1CCN(c2nn3cc(-c4ccc(Cl)cc4)nc3s2)CC1]

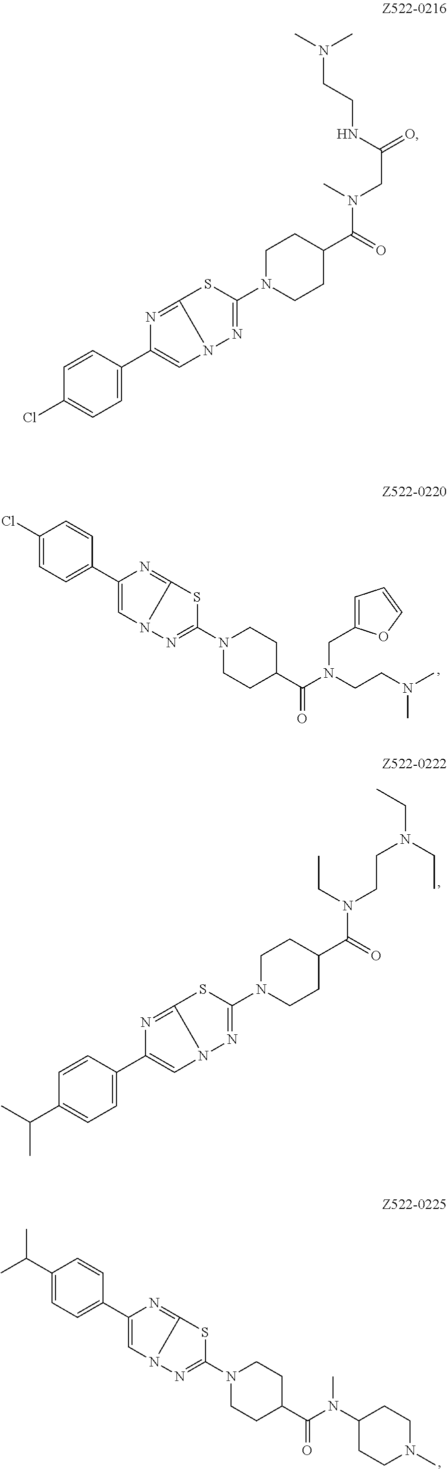

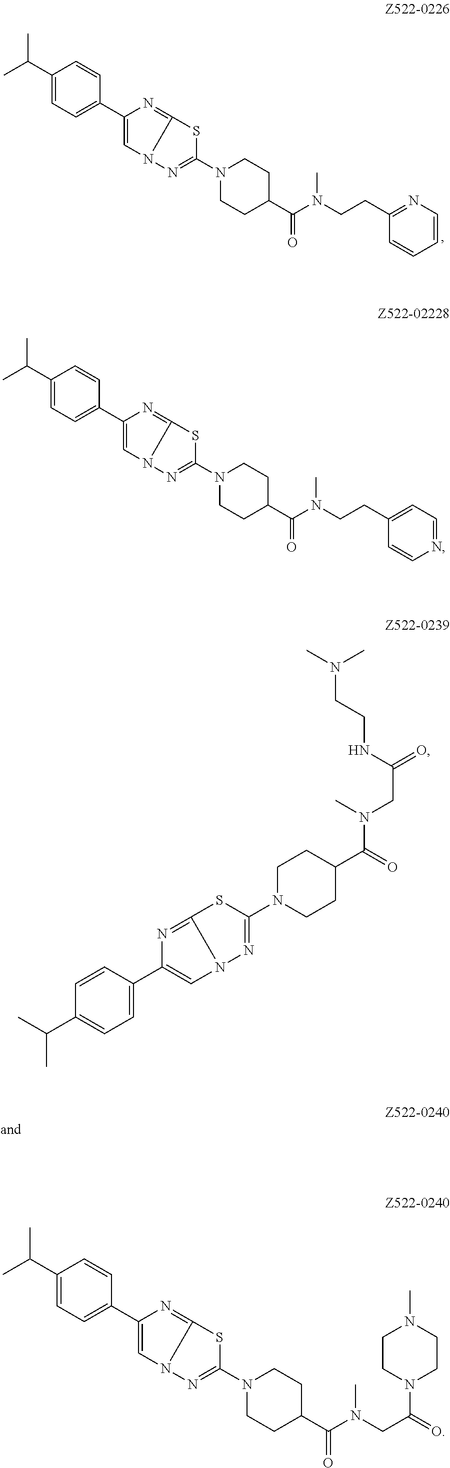 [SMILES: CC(C)c1ccc(-c2cn3nc(N4CCC(C(=O)N(C)CCc5ccccn5)CC4)sc3n2)cc1 ; CC(C)c1ccc(-c2cn3nc(N4CCC(C(=O)N(C)CCc5ccncc5)CC4)sc3n2)cc1 ; CC(C)c1ccc(-c2cn3nc(N4CCC(C(=O)N(C)CC(=O)NCCN(C)C)CC4)sc3n2)cc1 ; CC(C)c1ccc(-c2cn3nc(N4CCC(C(=O)N(C)CC(=O)N5CCN(C)CC5)CC4)sc3n2)cc1]

9. The pharmaceutical composition according to claim 7, selected from the group consisting of Z522-166, Z522-0170, Z522-186, Z522-0218, Z522-224, Z522-0237, Z522-0238, and Z522-0241.

10. The pharmaceutical composition according to claim 8, selected from the group consisting of Z522-202, Z522-220, Z522-222, Z522-225, and Z522-0239.

11. A method for treating cancer comprising administering to an individual in need of such treatment an effective amount of a pharmaceutical composition according claim 1.

12. A method according to claim 11, wherein the cancer is colon cancer.

13. A method of preparation of compounds of formula 9, comprising the following reaction scheme: 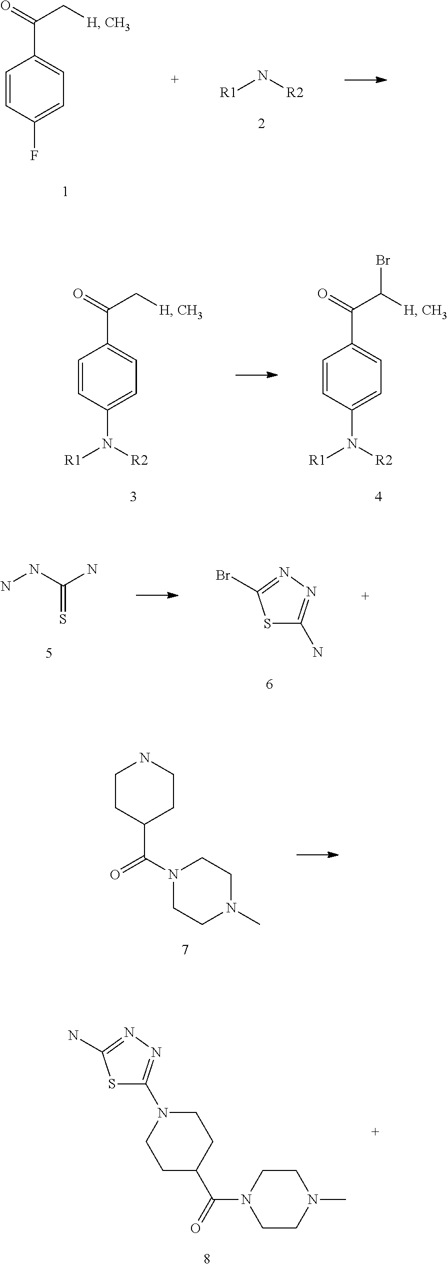 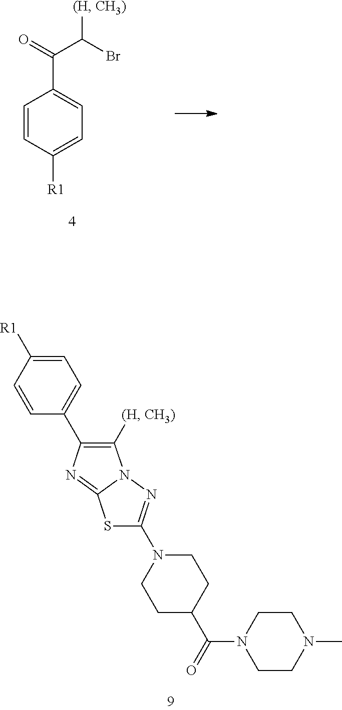
wherein R₁ and R₂ are independently H, straight or branched C₁-C₅ alkyl, or straight or branched C₁-C₅—OH.

14. A method of preparation of compounds of formula 9, comprising the following reaction scheme: 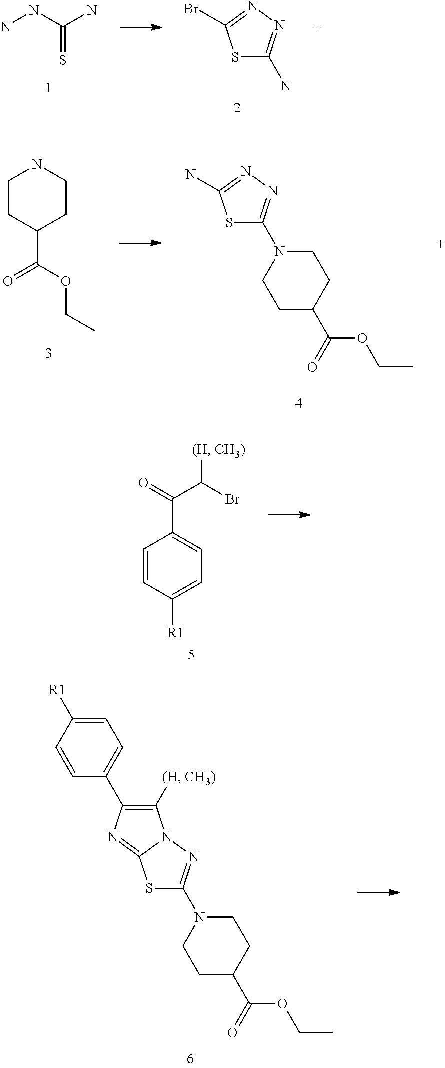 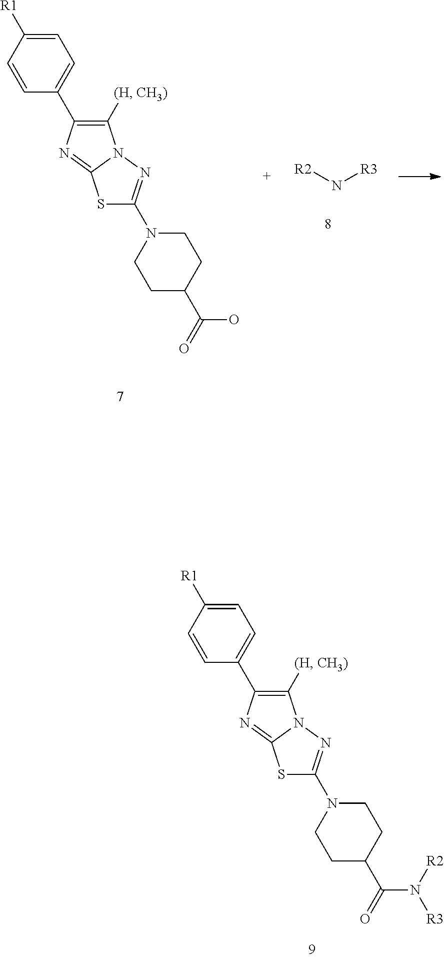
wherein R₁ and R₂ are independently H, straight or branched C₁-C₅ alkyl, or straight or branched C₁-C₅—OH; and R₃ is H, straight or branched C₁-C₅ alkyl, or (CH₂)ₙNR₁R₂.
